(19) United States
(12) Patent Application Publication
(10) Pub. No.: US 2026/0317368 A1
(43) Pub. Date: Oct. 15, 2026

(54) LATE-STAGE PEPTIDE CYCLIZATION TO FORM DISULFIDE MIMETICS

(71) Applicant: UNIVERSITY OF HONG KONG VERSITECH LIMITED, Hong Kong (CN)

(72) Inventors: Xuechen LI, Hong Kong (CN); Yisa XIAO, Hong Kong (CN)

(21) Appl. No.: 19/482,793

(22) PCT Filed: Apr. 25, 2024
(86) PCT No.: PCT/CN2024/089731

§ 371 Date: Nov. 10, 2025

Related U.S. Application Data
(60) Provisional application No. 63/467,583, filed on May 18, 2023.

(51) Int. Cl. C07K 1/107

(52) U.S. Cl. CPC C07K 1/1072

(57) ABSTRACT

Disclosed are methods for constructing disulfide mimetics for late-stage cyclization of peptides. The disclosed method provides late-stage cyclization via thioacetalization of peptides containing two cysteine residues to form thioacetal group. These bonds are stable under acidic, basic, and reduced conditions. The reaction can use a broad range of cyclic ketones even acetone as crosslinkers. Trifluoroacetic acid (TFA) alone can act as both the catalyst and the sole solvent, thus allowing most peptide sequences to be cyclized via the disclosed method. The disclosed method provides a number of benefits and advantages, such as the use of TFA as both a robust catalyst of the reaction and the sole solvent needed, the easy availability of acetone and of diverse cyclic ketones for use as the reactant, the good chemoselectivity and tolerance of native peptide to the reaction, and the fact that the resulting crosslinks are structural mimetics of disulfides.

### CROSS-REFERENCE TO RELATED APPLICATION

This application claims the benefit of and priority to U.S. Provisional Application No. 63/467,583 filed May 18, 2023, the entire content of which is incorporated herein by reference for all purpose in its entirety.

### FIELD OF THE INVENTION

The disclosed invention is generally in the field of protein and peptide crosslinking and specifically in the area of crosslinking peptides using late-stage disulfide mimetics.

### BACKGROUND OF THE INVENTION

Among the late-stage modification of peptides, cyclization is a vital strategy to enhance the conformation rigidity compared to linear peptides. The reduced flexibility could lock the peptide into the conformation and increase the stability and resistance to degradation by protease. Peptide disulfide cyclization is a powerful technique in drug development that involves the formation of a cyclic peptide through the formation of a disulfide bond between two cysteine residues in the peptide chain. This process results in a more stable and conformationally constrained molecule that exhibits improved pharmacological properties such as enhanced potency, selectivity, and stability. Peptide disulfide cyclization has gained significant attention in recent years due to its potential in developing novel therapeutics for various diseases including cancer, infectious diseases, and metabolic disorders. This approach has been successful in the development of several FDA-approved drugs such as insulin, oxytocin, and vasopressin. But the inherent instability of disulfide under reducing environments limits its broad applications and wide drug distribution.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### BRIEF SUMMARY OF THE INVENTION

Disclosed are methods for constructing disulfide mimetics for late-stage cyclization of peptides. Native disulfide bonds are not stable under reducing environments which limit its broad application. The disclosed late-stage cyclization via thioacetalization of peptides containing two cysteine residues to form thioacetal group which is stable under acidic, basic, and reduced conditions. Compared to prior methods, the disclosed thioacetalization of peptides can generate novel disulfide mimetics. The reaction can use a broad range of cyclic ketones even acetones as crosslinkers. Almost all peptide sequences can be used in the disclosed method since trifluoroacetic acid (TFA) alone can act as both the catalyst and the sole solvent.

The disclosed method provides a number of benefits and advantages, such as the use of TFA as both a robust catalyst of the reaction and the sole solvent needed, the easy availability of acetone and of diverse cyclic ketones for use as the reactant, the good chemoselectivity and tolerance of native peptide to the reaction, and the fact that the resulting crosslinks are structural mimetics of disulfides.

Disclosed are methods, and reagents for use therein, for late-stage cyclization of a peptide. Generally, the method includes maintaining a reaction mixture at a sufficient temperature for a sufficient period of time to form a product. Generally, the reaction mixture includes the peptide containing two or more cysteine residues, a cyclic ketone reagent or acetone, and a solvent. Generally, the product includes a thioacetalated peptide, where the thioacetalation couples two of the cysteine residues of the peptide.

In some forms, the peptide is linear or cyclic (including monocyclic, bicyclic, etc.). In some forms, the peptide is a random peptide or a peptide drug. In some forms, the peptide is formed by natural amino acids.

In some forms, the cyclic ketone reagent is a cyclic ketone reagent having the structure of:

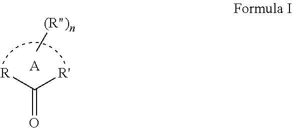

where R and R′ are independently an alkyl (such as a C1-C6 alkyl) and [symbol] is absent, or R and R′ together form a cyclic moiety A, where A is a cycloalkyl, a cycloalkenyl, a cycloalkynyl, an aryl, a polyaryl, a heteroaryl, a heteropolyaryl, or a heterocyclic, where R″ represents hydrogen or a substituent on the cyclic moiety A, and each occurrence of R″ is independently a benzyl, an allyl, an alkyl (such as a C1-C6 alkyl), a halogen, —CN, —CF₃, —NO₂, an alkoxy, or an aryl (such as a phenyl), and where n is an integer from 0 to 10, from 0 to 8, from 0 to 6, from 0 to 4, or from 0 to 2. When R″ is an alkyl, the alkyl can be a substituted or unsubstituted alkyl, and the substituent(s), when present, can be any substituent(s) disclosed herein, such as a halide, an azido, an alkynyl (such as —CCH or —CH₂CCH).

In some forms, R and R′ are independently an alkyl, such as methyl. In some forms, R and R′ are independently an alkyl, such as methyl, and [symbol] is absent. In some forms, A is cyclobutane, azetidine, cyclopentane, fluorenone, cyclohexane, or piperidine.

In some forms, the solvent is trifluoroacetic acid. In some forms, trifluoroacetic acid acts as catalyst and is the only solvent in the reaction mixture.

In some forms, the reaction mixture is maintained at a temperature ranging from 20° C. to 35° C., such as about 30° C., for a time period up to 1 hour, up to 2 hours, up to 3 hours, ranging from 10 mins to 1 hour, from 20 mins to 2 hours, or from 30 mins to 3 hours.

In some forms, the peptide and the cyclic ketone reagent have a molar ratio (peptide:cyclic ketone reagent) ranging from 0.1 to 1, such as about 0.2.

In some forms, the cyclic ketone reagent has any one of the following structures:

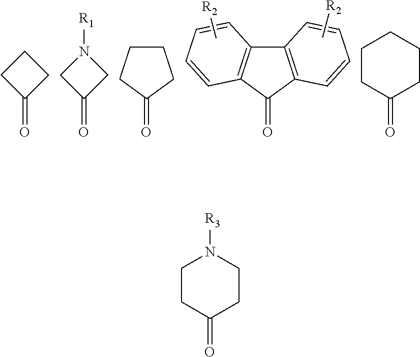

where R₁ and R₃ are an H, a benzyl, an allyl, an alkyl (such as a C1-C6 alkyl), and where each R₂ is independently an H, a benzyl, an allyl, an alkyl (such as a C1-C6 alkyl), a halogen, —CN, —CF₃, —NO₂, an alkoxy, or an aryl. When any of R₁-R3 is an alkyl, the alkyl can be a substituted or unsubstituted alkyl, and the substituent(s), when present, can be any substituent(s) disclosed herein, such as a halide, an azido, or an alkynyl (such as —CCH or —CH₂CCH).

In some forms, the thioacetalated peptide has any one of the following structures:

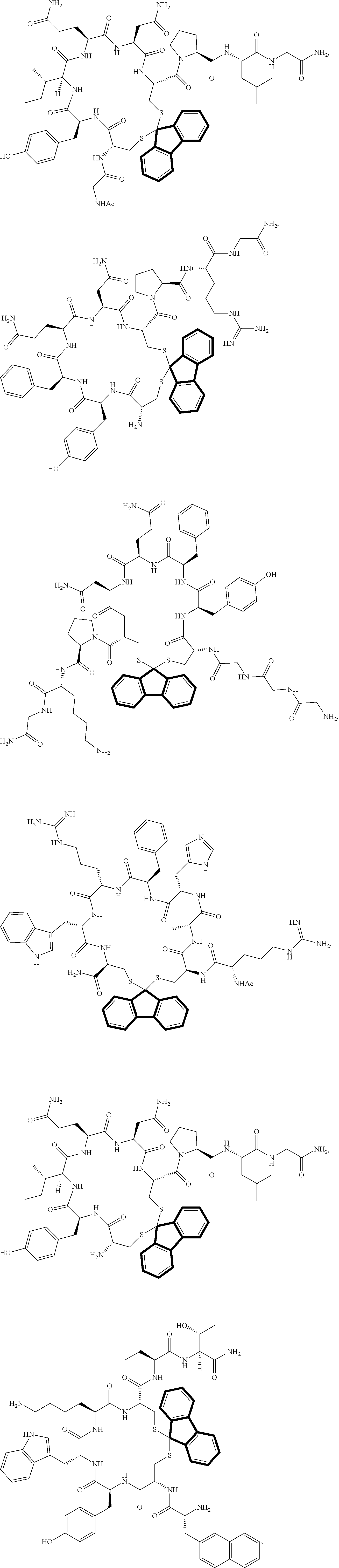

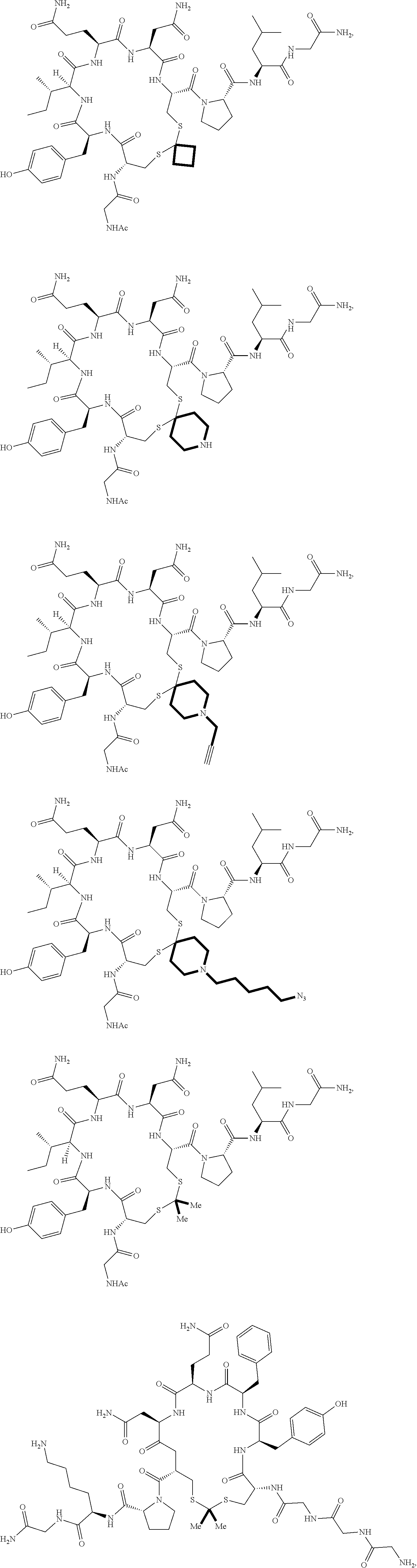

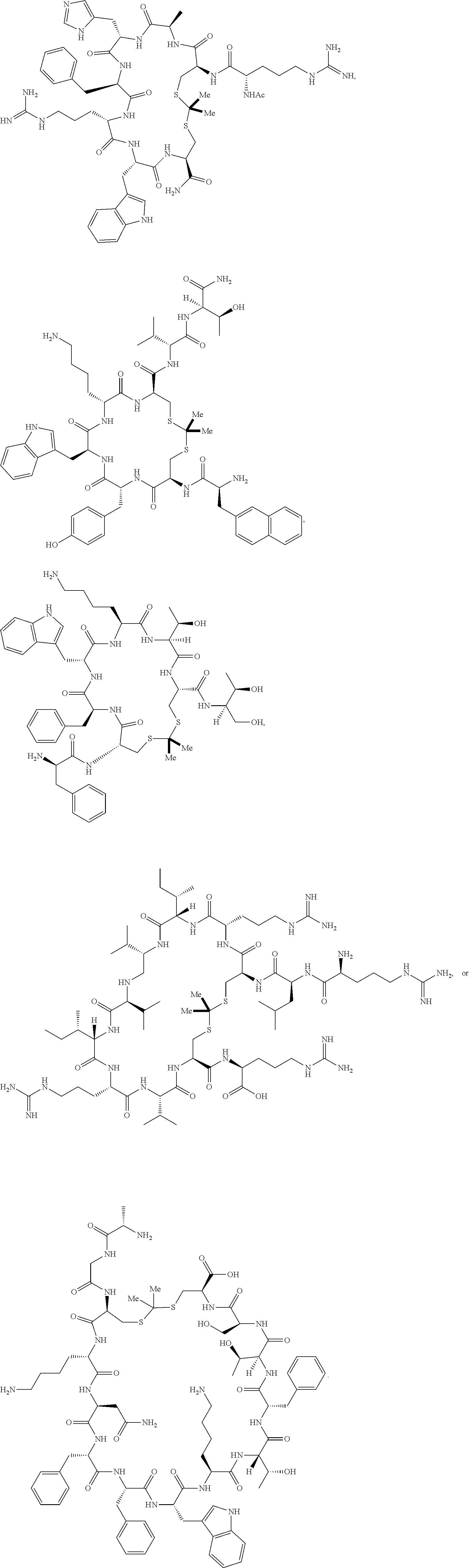

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings illustrate several embodiments of the disclosed method and compositions and together with the description, serve to explain the principles of the disclosed method and compositions.

FIG. 1 is a diagram of Scheme 2 (late-stage peptide cyclization with cyclic ketones).

FIG. 2 is a diagram of examples of late-stage peptide cyclization with different cyclic ketones.

FIG. 3 is a diagram of examples of late-stage peptide cyclization of native peptides with a cyclic ketone.

FIG. 4 is a diagram of Scheme 3 (late-stage peptide cyclization with acetone).

FIG. 5 is a diagram of examples of late-stage peptide cyclization of native peptides with acetone.

FIG. 6. UV (190-400 nm) and MS (250-3000 m/z) traces from UPLC-MS analysis of 2a (gradient 5-95% CH₃CN/H₂O containing 0.1% TFA over 5 min at a flow rate of 0.4 mL/min). ESI-MS calcd. for C₆₀H₇₉N₁₃O₁₄S₂, [M+H]⁺ m/z=1271.4, found 1270.5; [M+2H]²⁺ m/z=636.2, found 636.1.

FIG. 7. UV (190-400 nm) and MS (250-3000 m/z) traces from UPLC-MS analysis of 2b (gradient 5-95% CH₃CN/H₂O containing 0.1% TFA over 5 min at a flow rate of 0.4 mL/min). ESI-MS calcd. for C₃₀H₃₆N₆O₆S₂, [M+H]⁺ m/z=641.2, found 641.3.

FIG. 8. UV (190-400 nm) and MS (250-3000 m/z) traces from UPLC-MS analysis of 2c (gradient 10-50% CH₃CN/H₂O containing 0.1% TFA over 8 min at a flow rate of 0.4 mL/min). ESI-MS calcd. for C₅₉H₇₃N₁₅O₁₂S₂, [M+H]⁺ m/z=1249.4, found 1248.9; [M+2H]²⁺ m/z=625.2, found 625.0.

FIG. 9. UV (190-400 nm) and MS (250-3000 m/z) traces from UPLC-MS analysis of 2d (gradient 10-50% CH₃CN/H₂O containing 0.1% TFA over 8 min at a flow rate of 0.4 mL/min). ESI-MS calcd. for C₆₆H₈₃N₁₅O₁₅S₂, [M+H]⁺ m/z=1391.5, found 1391.9; [M+2H]²⁺ m/z=696.3, found 696.7.

FIG. 10. UV (190-400 nm) and MS (250-3000 m/z) traces from UPLC-MS analysis of 2e (gradient 10-50% CH₃CN/H₂O containing 0.1% TFA over 8 min at a flow rate of 0.4 mL/min). ESI-MS calcd. for C₆₂H₇₆N₁₈O₉S₂, [M+2H]²⁺ m/z=641.7, found 641.9; [M+3H]³⁺ m/z=428.1, found 428.2.

FIG. 11. UV (190-400 nm) and MS (250-3000 m/z) traces from UPLC-MS analysis of 2f (gradient 15-60% CH₃CN/H₂O containing 0.1% TFA over 8 min at a flow rate of 0.4 mL/min). ESI-MS calcd. for C₆₇H₇₇NO₁₀S₂, [M+H]⁺ m/z=1172.3, found 1171.7; [M+2H]⁺ m/z=586.7, found 586.6.

FIG. 12. UV (190-400 nm) and MS (250-3000 m/z) traces from UPLC-MS analysis of 2g (gradient 20-70% CH₃CN/H₂O containing 0.1% TFA over 8 min at a flow rate of 0.4 mL/min). ESI-MS calcd. for C₆₇H₇₇NO₁₀S₂, [M+H]⁺ m/z=1261.5, found 1260.8; [M+2H]⁺ m/z=631.2, found 631.3.

FIG. 13. UV (190-400 nm) and MS (300-2000 m/z) traces from UPLC-MS analysis of 2h (gradient 5-95% CH₃CN/H₂O containing 0.1% TFA over 5 min at a flow rate of 0.4 mL/min). ESI-MS calcd. for C₅₁H₇₇N₁₃O₁₄S₂, [M+H]⁺ m/z=1161.3, found 1160.6; [M+2H]⁺ m/z=581.1, found 581.1.

FIG. 14. UV (190-400 nm) and MS (250-3000 m/z) traces from UPLC-MS analysis of 2i (gradient 10-50% CH₃CN/H₂O containing 0.1% TFA over 8 min at a flow rate of 0.4 mL/min). ESI-MS calcd. for C₅₂H₈₀N₁₄O₁₄S₂, [M+H]⁺ m/z=1190.4, found 1189.8; [M+2H]⁺ m/z=595.7, found 595.6.

FIG. 15. UV (190-400 nm) and MS (250-3000 m/z) traces from UPLC-MS analysis of 2j (gradient 10-50% CH₃CN/H₂O containing 0.1% TFA over 8 min at a flow rate of 0.4 mL/min). ESI-MS calcd. for C₅₅H₈₂N₁₄O₁₄S₂, [M+Na]⁺ m/z=1249.9, found 1249.9; [M+H]⁺ m/z=1228.4, found 1227.8; [M+2H]²⁺ m/z=614.7, found 614.6.

FIG. 16. UV (190-400 nm) and MS (250-3000 m/z) traces from UPLC-MS analysis of 2k (gradient 10-60% CH₃CN/H₂O containing 0.1% TFA over 8 min at a flow rate of 0.4 mL/min). ESI-MS calcd. for C₅₇H₈₉N₁₇O₁₄S₂, [M+H]⁺ m/z=1301.5, found 1300.8; [M+2H]²⁺ m/z=651.2, found 651.1.

FIG. 17. UV (190-400 nm) and MS (250-3000 m/z) traces from UPLC-MS analysis of cyclo-(AcHN-GCYIQNCPLG-CONH₂) (gradient 5-95% CH₃CN/H₂O containing 0.1% TFA over 5 min at a flow rate of 0.4 mL/min). ESI-MS calcd. for C₅₀H₇₇N₁₃O₁₄S₂, [M+H]⁺ m/z=1149.3, found 1148.6; [M+2H]²⁺ m/z=575.1, found 575.1.

FIG. 18. UV (190-400 nm) and MS (250-3000 m/z) traces from UPLC-MS analysis of 3b (gradient 5-95% CH₃CN/H₂O containing 0.1% TFA over 5 min at a flow rate of 0.4 mL/min). ESI-MS calcd. for C₅₆H₈₁N₁₅O₁₅S₂, [M+H]⁺ m/z=1269.4, found 1270.2; [M+2H]⁺ m/z=635.2, found 635.6.

FIG. 19. UV (190-400 nm) and MS (250-3000 m/z) traces from UPLC-MS analysis of 3c (gradient 5-95% CH₃CN/H₂O containing 0.1% TFA over 5 min at a flow rate of 0.4 mL/min). ESI-MS calcd. for C₅₂H₇₄N₁₈O₉S₂, [M+H+TFA]⁺ m/z=1274.4, found 1273.7; [M+H]⁺ m/z=1160.3, found 1159.5; [M+2H]²⁺ m/z=580.7, found 580.6; [M+3H]³⁺ m/z=387.4, found 387.6.

FIG. 20. UV (190-400 nm) and MS (250-3000 m/z) traces from UPLC-MS analysis of 3d (gradient 5-95% CH₃CN/H₂O containing 0.1% TFA over 5 min at a flow rate of 0.4 mL/min). ESI-MS calcd. for C₅₇H₇₅N₁₁O₁₀S₂, [M+H]⁺ m/z=1139.4, found 1138.6; [M+2H]²⁺ m/z=570.2, found 570.0.

FIG. 21. UV (190-400 nm) and MS (300-2000 m/z) traces from UPLC-MS analysis of 3e (gradient 5-95% CH₃CN/H₂O containing 0.1% TFA over 5 min at a flow rate of 0.4 mL/min). ESI-MS calcd. for C₅₂H₇₂N₁₀O₁₀S₂, [M+H]⁺ m/z=1062.32, found 1061.7; [M+2H]²⁺ m/z=531.6, found 531.7.

FIG. 22. UV (190-400 nm) and MS (250-3000 m/z) traces from UPLC-MS analysis of 3f (gradient 5-95% CH₃CN/H₂O containing 0.1% TFA over 5 min at a flow rate of 0.4 mL/min). ESI-MS calcd. for C₆₆H₁₂₄N₂₄O₁₃S₂, [M+2H]²⁺ m/z=763.9, found 764.0; [M+3H]³⁺ m/z=509.6, found 509.8; [M+3H]³⁺ m/z=382.5, found 382.8.

FIG. 23. UV (190-400 nm) and MS (250-3000 m/z) traces from UPLC-MS analysis of 3g (gradient 5-95% CH₃CN/H₂O containing 0.1% TFA over 5 min at a flow rate of 0.4 mL/min). ESI-MS calcd. for C₇₉H₁₁₀N₁₈O₁₉S₂, [M+H]⁺ m/z=1680.9, found 1680.3; [M+2H]²⁺ m/z=840.9, found 840.9; [M+3H]³⁺ m/z=560.9, found 561.0.

### DETAILED DESCRIPTION OF THE INVENTION

The disclosed method and compositions can be understood more readily by reference to the following detailed description of particular embodiments and the Example included therein and to the Figures and their previous and following description.

Disclosed are methods for constructing disulfide mimetics for late-stage cyclization of peptides. Native disulfide bonds are not stable under reducing environments which limit its broad application. The disclosed late-stage cyclization via thioacetalization of peptides containing two cysteine residues to form thioacetal group which is stable under acidic, basic, and reduced conditions. Compared to prior methods, the disclosed thioacetalization of peptides can generate novel disulfide mimetics. The reaction can use a broad range of cyclic ketones even acetones as crosslinkers. Almost all peptide sequences can be used in the disclosed method since trifluoroacetic acid (TFA) alone can act as both the catalyst and the sole solvent.

The disclosed method provides a number of benefits and advantages, such as the use of TFA as both a robust catalyst of the reaction and the sole solvent needed, the easy availability of acetone and of diverse cyclic ketones for use as the reactant, the good chemoselectivity and tolerance of native peptide to the reaction, and the fact that the resulting crosslinks are structural mimetics of disulfides.

To overcome the deficiency of native disulfide bonds, there have been various methods of stapling based on cysteine peptides which are high in reactivity and selectivity to form disulfide bond mimetics. Symmetrical linkers such as dichloroacetone (DCA), dichloroacetophenone, di-bromo benzylic linkers, or di-leaving group substituted aryl linkers via S-alkylation or S-arylation are used to achieve stapling. Recently, Cramer and co-workers utilized CH₂I₂ reagent to react with cysteine peptide to form a thiocarbenium ion intermediate that undergoes conversion to methylene thioacetal, which eliminates the reductive liability of the disulfide group and enhanced structural stability. In contrast, in the disclosed methods, cyclic ketones and acetone can be applied as crosslinker agents for late-stage peptide cyclization based on cysteine residues under trifluoroacetic acid (TFA) conditions, generating a series of thioacetal disulfides mimetics with good chemoselectivity and tolerance of native peptides.

An exemplary general procedure of late-stage cyclization to form disulfide mimetics with cyclic ketones or acetone is shown in Scheme 1, where R¹-R³ are defined as follows:
each of R₁, R₃ is independently H, Bn, Allyl, or C₁-C₆ alkyl;
R₂ is a mono-substituted or multiply-substituted fluorenone, such as H, halogen, CN, CF₃, NO₂, Alkyl, alkoxy, or aryl.

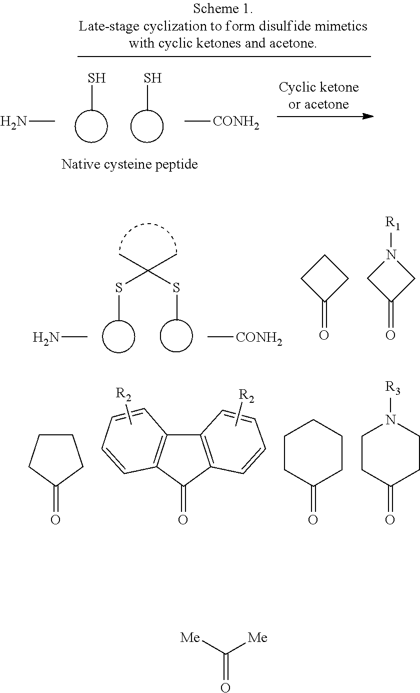

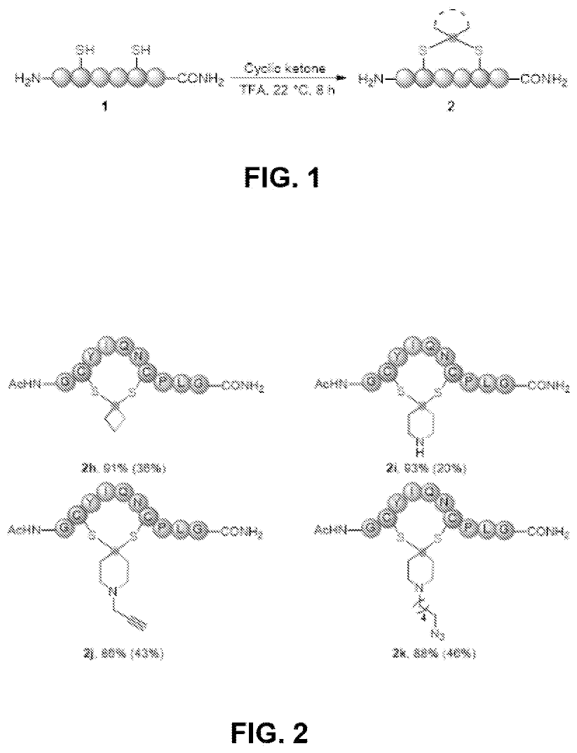

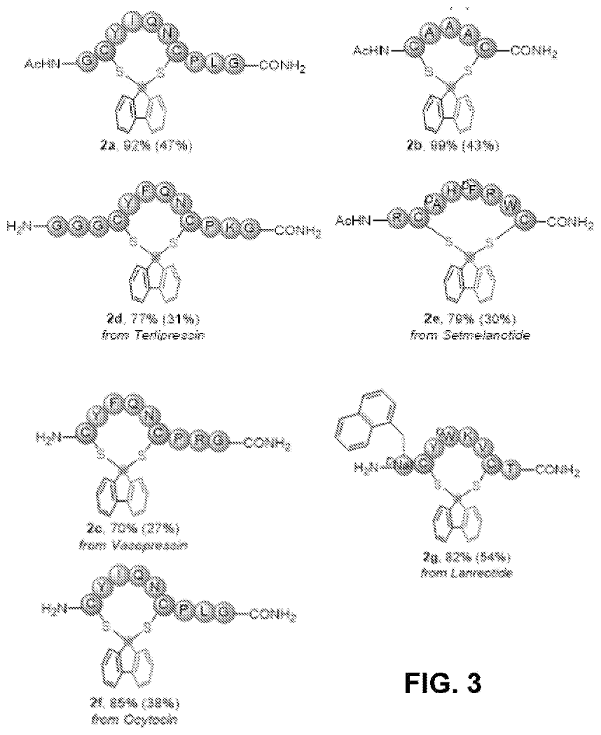

An exemplary general method of late-stage peptide cyclization is shown in Scheme 2 (FIG. 1). Purified native peptide sequence such as linear Vasopressin, Terlipressin, Setmelanotide, Oxytocin or Lanreotide after SPPS were dissolved in trifluoroacetic acid (TFA) with a final concentration of 10 mM. Cyclic ketone (20.0 equiv) with a concentration of 200 mM was added to the solution and the reaction was stirred at room temperature around 8 h. After finishing the reaction, the solvent was blown off under a stream of N₂, and then decanting diethyl ether. The residue was dissolved in the mix solvent of acetonitrile (ACN) and water (H₂O) then subjected to preparative HPLC purification. After lyophilization, the corresponding product was obtained. Here, the detailed synthetic procedures of late-stage peptide modification with cyclic ketones were described as follow as examples.

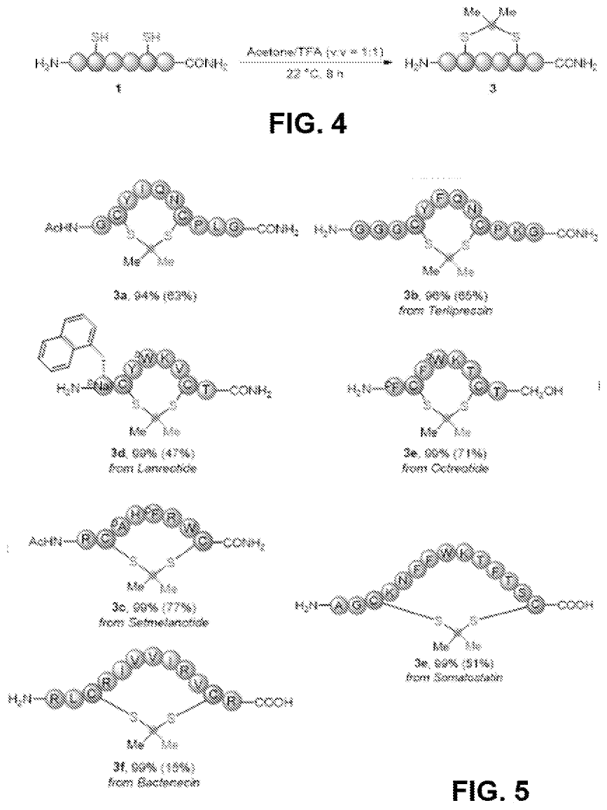

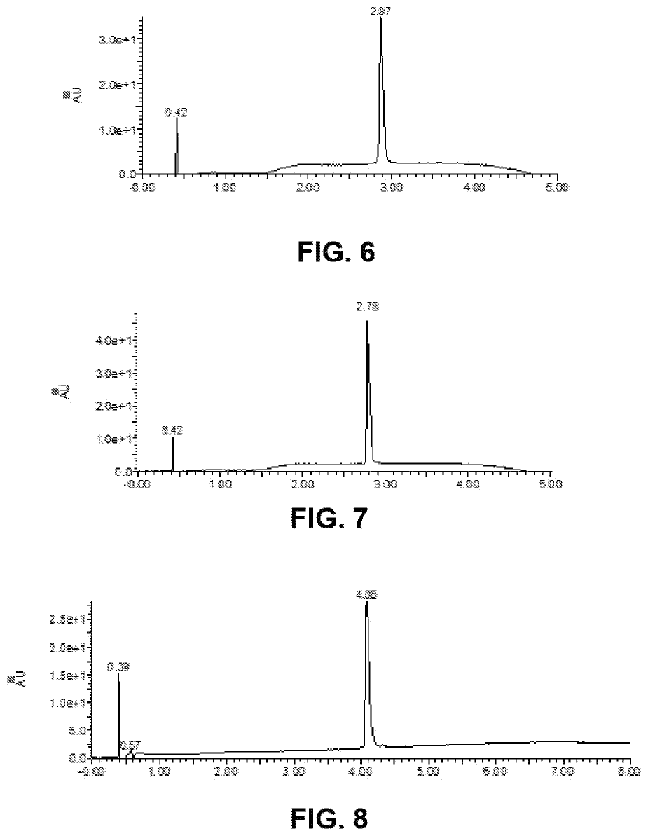

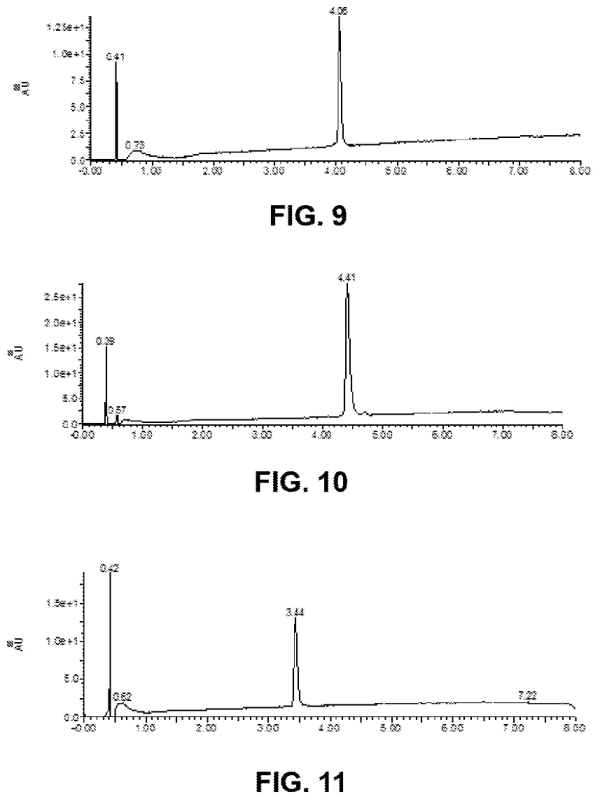

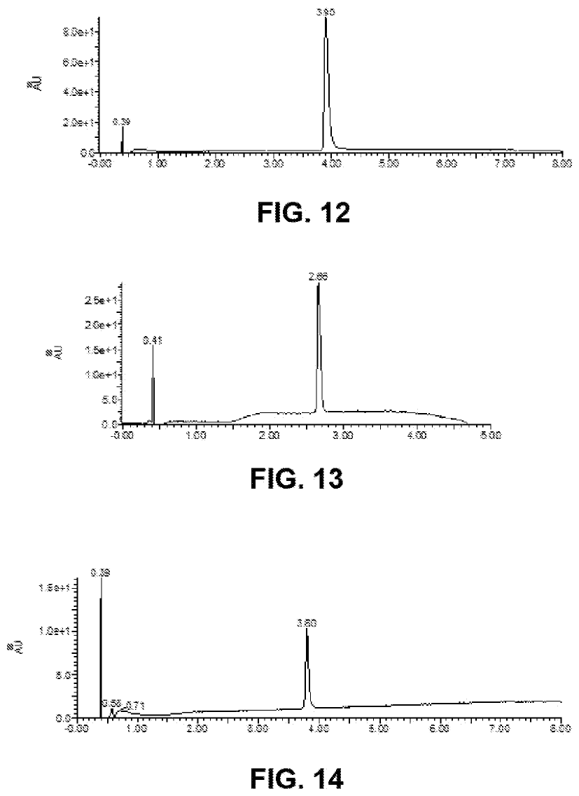

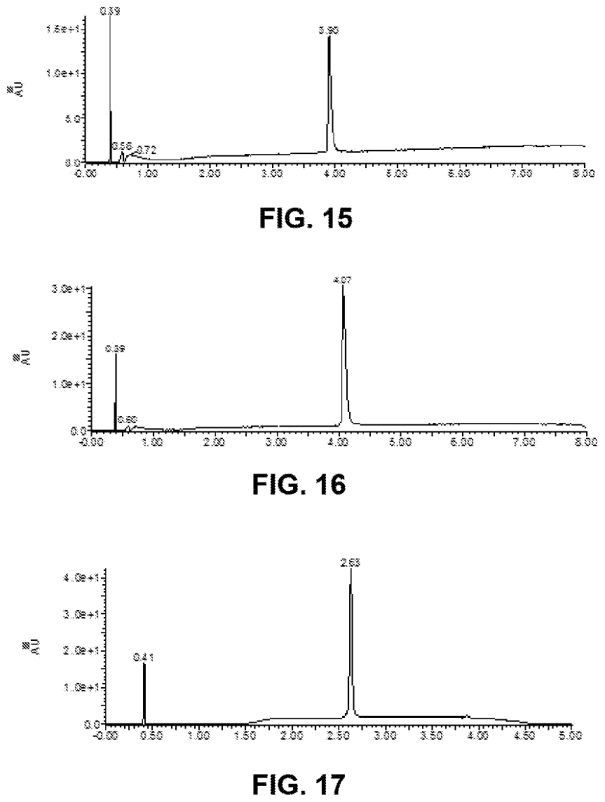

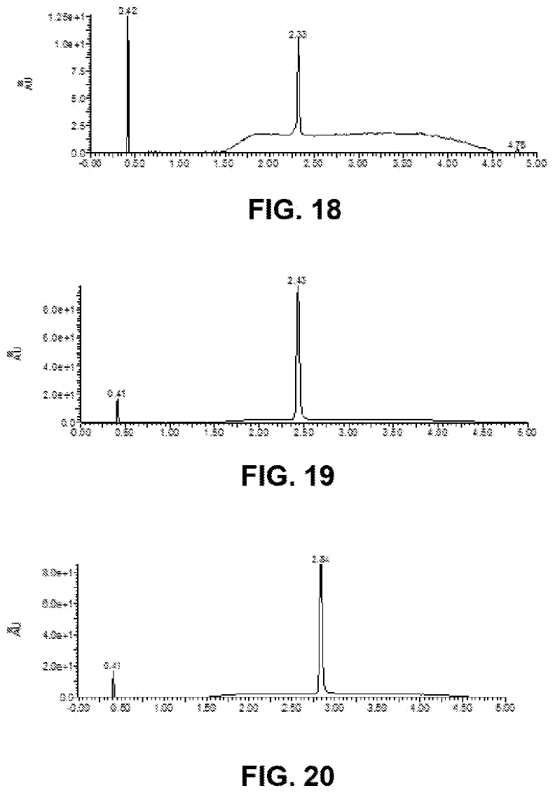

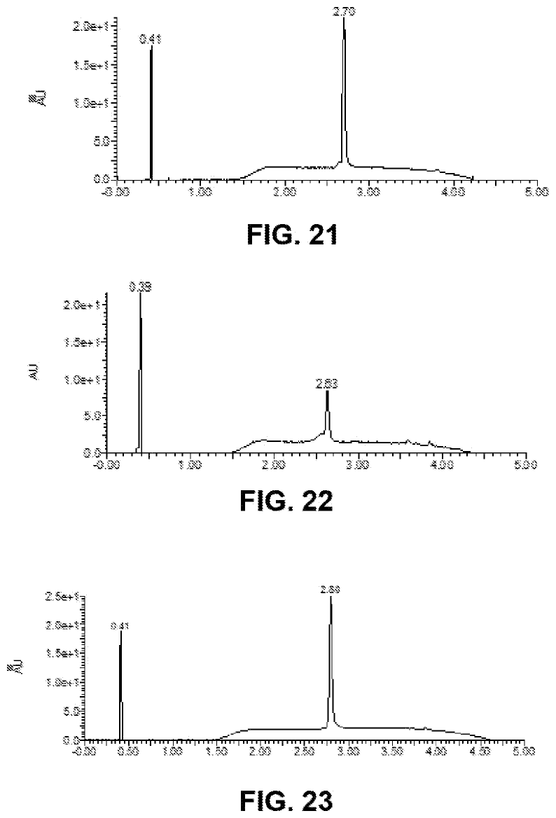

Another exemplary general method of late-stage peptide cyclization with acetone is shown in Scheme 3 (FIG. 4). Purified native peptide sequence such as Terlipressin, Setmelanotide, Lanreotide, Octreotide, Bactenecin and Somatostatin after SPPS was dissolved in a mixture solvent of TFA and acetone (v:v=1:1) with a final concentration of 10 mM. The reaction was stirred at room temperature around 8 h. After finishing the reaction, the solvent was blown off under a stream of N₂, and then crude peptide product was precipitated by diethyl ether. The residue was dissolved in the mix solvent of acetonitrile (ACN) and water (H₂O) then subjected to preparative HPLC purification. After lyophilization the target product was obtained. Here, the detailed synthetic procedures of late-stage peptide modification with cyclic ketones were described as follows as examples.

Disclosed are methods, and reagents for use therein, for late-stage cyclization of a peptide. Generally, the method includes maintaining a reaction mixture at a sufficient temperature for a sufficient period of time to form a product. Generally, the reaction mixture includes the peptide containing two or more cysteine residues, a cyclic ketone reagent or acetone, and a solvent. Generally, the product includes a thioacetalated peptide, where the thioacetalation couples two of the cysteine residues of the peptide.

In some forms, the peptide is linear or cyclic (including monocyclic, bicyclic, etc.). In some forms, the peptide is a random peptide or a peptide drug. In some forms, the peptide is formed by natural amino acids.

In some forms, the cyclic ketone reagent or acetone is a cyclic ketone reagent having the structure of:

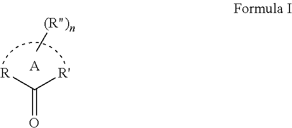

where R and R′ are independently an alkyl (such as a C₁-C₆ alkyl) and [symbol] is absent, or R and R′ together form a cyclic moiety A, where A is a cycloalkyl, a [symbol] cycloalkenyl, a cycloalkynyl, an aryl, a polyaryl, a heteroaryl, a heteropolyaryl, or a heterocyclic, where R″ represents hydrogen or a substituent on the cyclic moiety A, and each occurrence of R″ is independently a benzyl, an allyl, an alkyl (such as a C1-C6 alkyl), a halogen, —CN, —CF₃, —NO₂, an alkoxy, or an aryl (such as a phenyl), and where n is an integer from 0 to 10, from 0 to 8, from 0 to 6, from 0 to 4, or from 0 to 2. When R″ is an alkyl, the alkyl can be a substituted or unsubstituted alkyl, and the substituent(s), when present, can be any substituent(s) disclosed herein, such as a halide, an azido, an alkynyl (such as —CCH or —CH₂CCH).

In some forms, R and R′ are independently an alkyl, such as methyl. In some forms, R and R′ are independently an alkyl, such as methyl, and [symbol] is absent. In some forms, A is cyclobutane, azetidine, cyclopentane, fluorenone, cyclohexane, or piperidine.

In some forms, the solvent is trifluoroacetic acid. In some forms, trifluoroacetic acid acts as catalyst and is the only solvent in the reaction mixture.

In some forms, the reaction mixture is maintained at a temperature ranging from 20° C. to 35° C., such as about 30° C., for a time period up to 1 hour, up to 2 hours, up to 3 hours, ranging from 10 mins to 1 hour, from 20 mins to 2 hours, or from 30 mins to 3 hours.

In some forms, the peptide and the cyclic ketone reagent have a molar ratio (peptide:cyclic ketone reagent) ranging from 0.1 to 1, such as about 0.2.

In some forms, the cyclic ketone reagent has any one of the following structures:

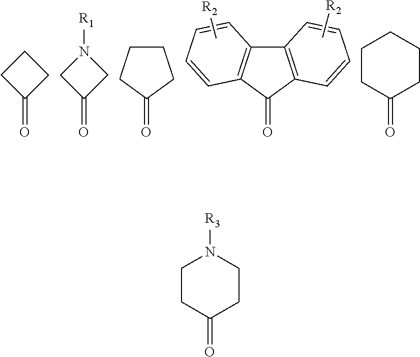

where R¹ and R³ are an H, a benzyl, an allyl, an alkyl (such as a C1-C6 alkyl), and where each R² is independently an H, a benzyl, an allyl, an alkyl (such as a C1-C6 alkyl), a halogen, —CN, —CF₃, —NO₂, an alkoxy, or an aryl. When any of R₁-R3 is an alkyl, the alkyl can be a substituted or unsubstituted alkyl, and the substituent(s), when present, can be any substituent(s) disclosed herein, such as a halide, an azido, or an alkynyl (such as —CCH or —CH₂CCH).

In some forms, the thioacetalated peptide has any one of the following structures:

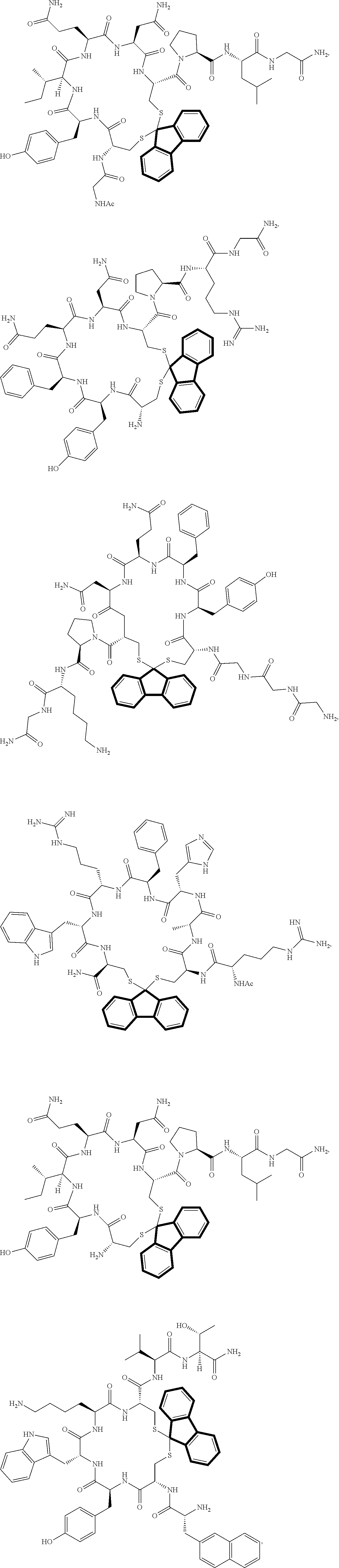

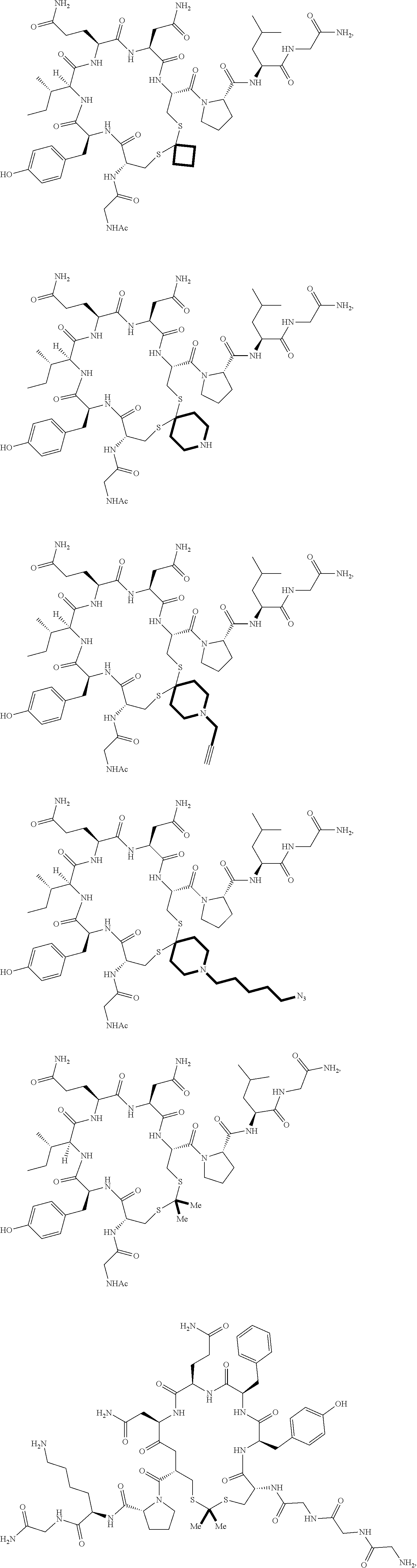

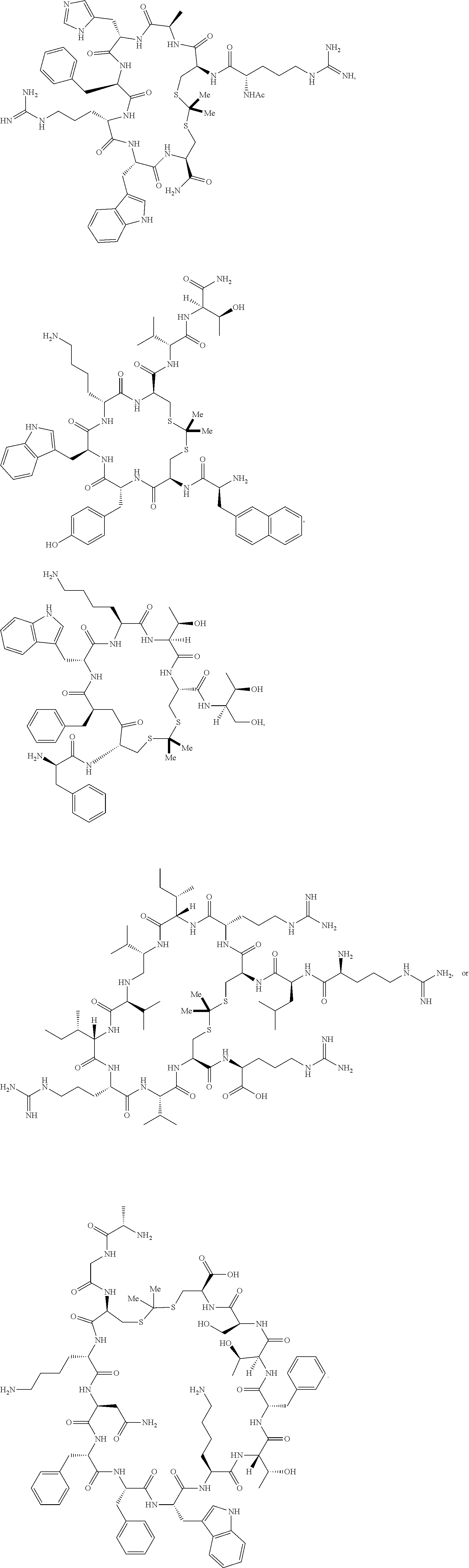

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid. Such a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, and an amino acid can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

“Alkyl,” as used herein, refers to the radical of saturated aliphatic groups, including straight-chain alkyl groups, branched-chain alkyl, and cycloalkyl (alicyclic). In some forms, a straight chain or branched chain alkyl has 30 or fewer carbon atoms in its backbone (e.g., C₁-C₃₀ for straight chains, C₃-C₃₀ for branched chains), 20 or fewer, 15 or fewer, or 10 or fewer. Alkyl includes methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Likewise, a cycloalkyl is a non-aromatic carbon-based ring composed of at least three carbon atoms, such as a nonaromatic monocyclic or nonaromatic polycyclic ring containing 3-30 carbon atoms, 3-20 carbon atoms, or 3-10 carbon atoms in their ring structure, and have 5, 6 or 7 carbons in the ring structure. Cycloalkyls containing a polycyclic ring system can have two or more non-aromatic rings in which two or more carbons are common to two adjoining rings (i.e., “fused cycloalkyl rings”). Examples of cycloalkyl groups include, but are not limited to, cyclopropyl, cyclobutyl, cyclopentyl, cyclohexyl, cycloheptyl, cyclooctanyl, etc.

“Substituted alkyl” refers to alkyl moieties having one or more substituents replacing a hydrogen on one or more carbons of the hydrocarbon backbone. Such substituents can be any substituents described above, e.g., halogen (such as fluorine, chlorine, bromine, or iodine), hydroxyl, carbonyl (such as a carboxyl, alkoxycarbonyl, formyl, or an acyl), thiocarbonyl (such as a thioester, a thioacetate, or a thioformate), aryl, alkoxyl, aralkyl, phosphonium, phosphanyl, phosphonyl, phosphoryl, phosphate, phosphonate, a phosphinate, amino, amido, amidine, imine, cyano, nitro, azido, oxo, sulfhydryl, thiol, alkylthio, silyl, sulfinyl, sulfate, sulfonate, sulfamoyl, sulfonamido, sulfonyl, heterocyclyl, an aromatic or heteroaromatic moiety. —NRR′, wherein R and R′ are independently hydrogen, alkyl, or aryl, and wherein the nitrogen atom is optionally quaternized; —SR, wherein R is a phosphonyl, a sulfinyl, a silyl a hydrogen, an alkyl, or an aryl; —CN; —NO₂; —COOH; carboxylate; —COR, —COOR, or —CON(R)₂, wherein R is hydrogen, alkyl, or aryl; imino, silyl, ether, haloalkyl (such as —CF3, —CH₂—CF₃, —CCl₃); —CN; —NCOCOCH₂CH₂; —NCOCOCHCH; and —NCS; and combinations thereof.

It will be understood by those skilled in the art that the moieties substituted on the hydrocarbon chain can themselves be substituted, if appropriate. For instance, the substituents of a substituted alkyl may include halogen, hydroxy, nitro, thiols, amino, aralkyl, azido, imino, amido, phosphonium, phosphanyl, phosphoryl (including phosphonate and phosphinate), oxo, sulfonyl (including sulfate, sulfonamido, sulfamoyl and sulfonate), and silyl groups, as well as ethers, alkylthios, carbonyls (including ketones, aldehydes, carboxylates, and esters), haloalkyls, —CN and the like. Cycloalkyls can be substituted in the same manner.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths.

“Heteroalkyl,” as used herein, refers to straight or branched chain, or cyclic carbon-containing alkyl radicals, or combinations thereof, containing at least one heteroatom on the carbon backbone. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “alkenyl” as used herein is a hydrocarbon group of from 2 to 24 carbon atoms and structural formula containing at least one carbon-carbon double bond. Alkenyl groups include straight-chain alkenyl groups, branched-chain alkenyl, and cycloalkenyl. A cycloalkenyl is a non-aromatic carbon-based ring composed of at least three carbon atoms and at least one carbon-carbon double bond, such as a nonaromatic monocyclic or nonaromatic polycyclic ring containing 3-30 carbon atoms and at least one carbon-carbon double bond, 3-20 carbon atoms and at least one carbon-carbon double bond, or 3-10 carbon atoms and at least one carbon-carbon double bond in their ring structure, and have 5, 6 or 7 carbons and at least one carbon-carbon double bond in the ring structure. Cycloalkenyls containing a polycyclic ring system can have two or more non-aromatic rings in which two or more carbons are common to two adjoining rings (i.e., “fused cycloalkenyl rings”) and contain at least one carbon-carbon double bond.

Asymmetric structures such as (AB)C═C(C′D) are intended to include both the E and Z isomers. This may be presumed in structural formulae herein wherein an asymmetric alkene is present, or it may be explicitly indicated by the bond symbol C. The term “alkenyl” as used throughout the specification, examples, and claims is intended to include both “unsubstituted alkenyls” and “substituted alkenyls,” the latter of which refers to alkenyl moieties having one or more substituents replacing a hydrogen on one or more carbons of the hydrocarbon backbone. The term “alkenyl” also includes “heteroalkenyl.”

The term “substituted alkenyl” refers to alkenyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, oxo, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkenyl,” as used herein, refers to straight or branched chain, or cyclic carbon-containing alkenyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkenyl group” is a cycloalkenyl group where at least one of the carbon atoms of the ring is substituted with a heteroatom such as, but not limited to, nitrogen, oxygen, sulphur, or phosphorus.

The term “alkynyl group” as used herein is a hydrocarbon group of 2 to 24 carbon atoms and a structural formula containing at least one carbon-carbon triple bond. Alkynyl groups include straight-chain alkynyl groups, branched-chain alkynyl, and cycloalkynyl. A cycloalkynyl is a non-aromatic carbon-based ring composed of at least three carbon atoms and at least one carbon-carbon triple bond, such as a nonaromatic monocyclic or nonaromatic polycyclic ring containing 3-30 carbon atoms and at least one carbon-carbon triple bond, 3-20 carbon atoms and at least one carbon-carbon triple bond, or 3-10 carbon atoms and at least one carbon-carbon triple bond in their ring structure, and have 5, 6 or 7 carbons and at least one carbon-carbon triple bond in the ring structure. Cycloalkynyls containing a polycyclic ring system can have two or more non-aromatic rings in which two or more carbons are common to two adjoining rings (i.e., “fused cycloalkynyl rings”) and contain at least one carbon-carbon triple bond. Asymmetric structures such as (AB)C≡C(C″D) are intended to include both the E and Z isomers. This may be presumed in structural formulae herein wherein an asymmetric alkyne is present, or it may be explicitly indicated by the bond symbol C. The term “alkynyl” as used throughout the specification, examples, and claims is intended to include both “unsubstituted alkynyls” and “substituted alkynyls,” the latter of which refers to alkynyl moieties having one or more substituents replacing a hydrogen on one or more carbons of the hydrocarbon backbone. The term “alkynyl” also includes “heteroalkynyl.”

The term “substituted alkynyl” refers to alkynyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkynyl,” as used herein, refers to straight or branched chain, or cyclic carbon-containing alkynyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkynyl group” is a cycloalkynyl group where at least one of the carbon atoms of the ring is substituted with a heteroatom such as, but not limited to, nitrogen, oxygen, sulphur, or phosphorus.

“Aryl,” as used herein, refers to C₅-C₂₆-membered aromatic or fused aromatic ring systems. Examples of aromatic groups are benzene, naphthalene, anthracene, phenanthrene, chrysene, pyrene, corannulene, coronene, etc.

The term “substituted aryl” refers to an aryl group, wherein one or more hydrogen atoms on one or more aromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

“Heterocycle” and “heterocyclyl” are used interchangeably, and refer to a cyclic radical attached via a ring carbon or nitrogen atom of a non-aromatic monocyclic or polycyclic ring containing 3-30 ring atoms, 3-20 ring atoms, 3-10 ring atoms, or 5-6 ring atoms, where each ring contains carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Heterocycles can be a heterocycloalkyl, a heterocycloalkenyl, a heterocycloalkynyl, etc, such as piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₅-C₂₆-membered aromatic or fused aromatic ring systems, in which one or more carbon atoms on one or more aromatic ring structures have been substituted with a heteroatom. Suitable heteroatoms include, but are not limited to, oxygen, sulfur, and nitrogen. Examples of heteroaryl groups pyrrole, furan, thiophene, imidazole, oxazole, thiazole, triazole, tetrazole, pyrazole, pyridine, pyrazine, pyridazine and pyrimidine, and the like. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl.”

The term “substituted heteroaryl” refers to a heteroaryl group in which one or more hydrogen atoms on one or more heteroaromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

The term “polyaryl” refers to a chemical moiety that includes two or more fused aryl groups. When two or more fused heteroaryl groups are involved, the chemical moiety can be referred to as a “polyheteroaryl.”

The term “substituted polyaryl” refers to a polyaryl in which one or more of the aryls are substituted, with one or more substituents including, but not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof. When a polyheteroaryl is involved, the chemical moiety can be referred to as a “substituted polyheteroaryl.”

The term “cyclic ring” or “cyclic group” refers to a substituted or unsubstituted monocyclic ring or a substituted or unsubstituted polycyclic ring (such as those formed from single or fused ring systems), such as a substituted or unsubstituted cycloalkyl, a substituted or unsubstituted cycloalkenyl, a substituted or unsubstituted cycloalkynyl, or a substituted or unsubstituted heterocyclyl, that have from three to 30 carbon atoms, as geometric constraints permit. The substituted cycloalkyls, cycloalkenyls, cycloalkynyls, and heterocyclyls are substituted as defined above for the alkyls, alkenyls, alkynyls, and heterocyclyls, respectively.

The term “aralkyl” as used herein is an aryl group or a heteroaryl group having an alkyl, alkynyl, or alkenyl group as defined above attached to the aromatic group, such as an aryl, a heteroaryl, a polyaryl, or a polyheteroaryl. An example of an aralkyl group is a benzyl group.

The terms “thiol” are used interchangeably and are represented by —SR, where R can be a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, an amido, an amino, an alkoxy, an oxo, a phosphonyl, a sulfinyl, or a silyl, described above. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted, independently, with the substituents described above in the definition of “substituted.”

The numerical ranges disclose individually each possible number that such a range could reasonably encompass, as well as any sub-ranges and combinations of sub-ranges encompassed therein. For example, in a given range carbon range of C₃-C₉, the range also discloses C₃, C₄, C₅, C₆, C₇, C₈, and C₉, as well as any subrange between these numbers (for example, C₄-C₆), and any possible combination of ranges possible between these values. In yet another example, a given temperature range may be from about 25° C. to 30° C., where the range also discloses temperatures that can be selected independently from about 25, 26, 27, 28, 29, and 30° C., as well as any range between these numbers (for example, 26 to 28° C.), and any possible combination of ranges between these values.

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

“Oxo” refers to ═O.

The compounds and substituents can be substituted, independently, with the substituents described above in the definition of “substituted.”

Numerical ranges include ranges of 1 to 6. The ranges disclose individually each possible number that such a range could reasonably encompass, as well as any sub-ranges and combinations of sub-ranges encompassed therein. For example, in a given range carbon range of C₃-C₉, the range also discloses C₃, C₄, C₅, C₆, C₇, C₈, and C₉, as well as any subrange between these numbers (for example, C₄-C₆), and any possible combination of ranges possible between these values. In yet another example, a given temperature range may be from about 25° C. to 30° C., where the range also discloses temperatures that can be selected independently from about 25, 26, 27, 28, 29, and 30° C., as well as any range between these numbers (for example, 26 to 28° C.), and any possible combination of ranges between these values.

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

“Analog” as relates to a given compound, refers to another compound that is structurally similar, functionally similar, or both, to the specified compound. Structural similarity can be determined using any criterion known in the art, such as the Tanimoto coefficient that provides a quantitative measure of similarity between two compounds based on their molecular descriptors. Preferably, the molecular descriptors are 2D properties such as fingerprints, topological indices, and maximum common substructures, or 3D properties such as overall shape, and molecular fields. Tanimoto coefficients range between zero and one, inclusive, for dissimilar and identical pairs of molecules, respectively. A compound can be considered an analog of a specified compound, if it has a Tanimoto coefficient with the specified compound between 0.5 and 1.0, inclusive, preferably between 0.7 and 1.0, inclusive, most preferably between 0.85 and 1.0, inclusive. A compound is functionally similar to a specified, if it induces the same pharmacological effect, physiological effect, or both, as the specified compound. “Analog” can also refer to a modification including, but not limited to, hydrolysis, reduction, or oxidation products, of the disclosed compounds. Hydrolysis, reduction, and oxidation reactions are known in the art.

The disclosed compositions and methods can be further understood through the following numbered paragraphs.

1. A method of late-stage cyclization of a peptide comprising:
maintaining a reaction mixture at a sufficient temperature for a sufficient period of time to form a product,
wherein the reaction mixture comprises the peptide comprising two or more cysteine residues, a cyclic ketone reagent or acetone, and a solvent,
wherein the product comprises a thioacetalated peptide, and the thioacetalation couples two of the cysteine residues of the peptide.

2. The method of paragraph 1, wherein the peptide is linear or cyclic (including monocyclic, bicyclic, etc.)

3. The method of paragraph 1 or 2, wherein the peptide is a random peptide or a peptide drug.

4. The method of any one of paragraphs 1-3, wherein the peptide comprises natural amino acids.

5. The method of any one of paragraphs 1-4, wherein the cyclic ketone reagent is a cyclic ketone reagent having the structure of:

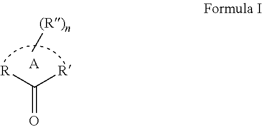

wherein R and R′ are independently an alkyl and [symbol] is absent, or R and R′ together form a cyclic moiety A,
wherein A is a cycloalkyl, a cycloalkenyl, a cycloalkynyl, an aryl, a polyaryl, a heteroaryl, a heteropolyaryl, or a heterocyclic,
wherein R″ represents hydrogen or a substituent on the cyclic moiety A, and each occurrence of R″ is independently a benzyl, an allyl, an alkyl (such as a C₁-C₆ alkyl), a halogen, —CN, —CF₃, —NO₂, an alkoxy, or an aryl, and
wherein n is an integer from 0 to 10, from 0 to 8, from 0 to 6, from 0 to 4, or from 0 to 2.

6. The method of paragraph 5, wherein R and R′ are independently an alkyl, such as methyl.

7. The method of paragraph 5 or 6, wherein A is cyclobutane, azetidine, cyclopentane, fluorenone, cyclohexane, or piperidine.

8. The method of any one of paragraphs 1-7, wherein the solvent is trifluoroacetic acid.

9. The method of paragraph 8, wherein trifluoroacetic acid acts as catalyst and/or is the only solvent in the reaction mixture.

10. The method of any one of paragraphs 1-9, wherein the reaction mixture is maintained at a temperature ranging from 20° C. to 35° C., such as about 30° C., for a time period up to 1 hour, up to 2 hours, up to 3 hours, ranging from 10 mins to 1 hour, from 20 mins to 2 hours, or from 30 mins to 3 hours.

11. The method of any one of paragraphs 1-10, wherein the peptide and the cyclic ketone reagent have a molar ratio (peptide:cyclic ketone reagent) ranging from 0.1 to 1, such as about 0.2.

12. The method of any one of paragraphs 1-11, wherein the cyclic ketone reagent has any one of the following structures:

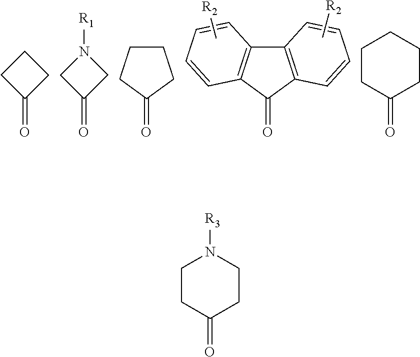

wherein R¹ and R³ are an H, a benzyl, an allyl, an alkyl (such as a C₁-C₆ alkyl), and
wherein each R² is independently an H, a benzyl, an allyl, an alkyl (such as a C1-C6 alkyl), a halogen, —CN, —CF₃, —NO₂, an alkoxy, or an aryl.

13. The method of any one of paragraphs 1-12, wherein the thioacetalated peptide has any one of the following structures:

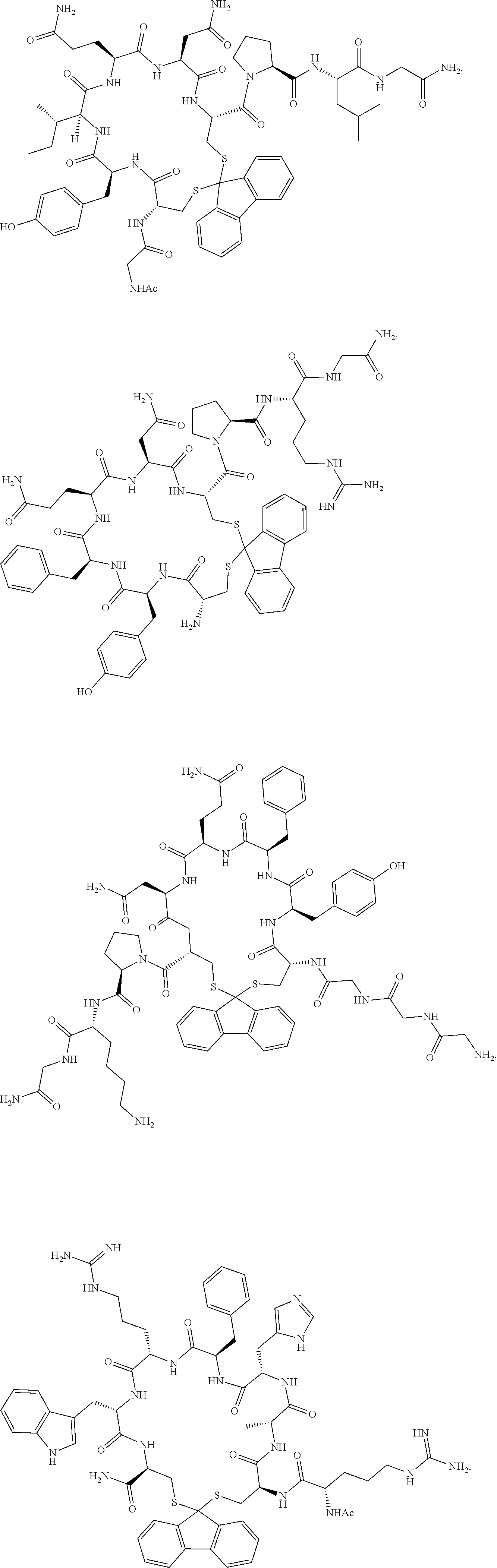

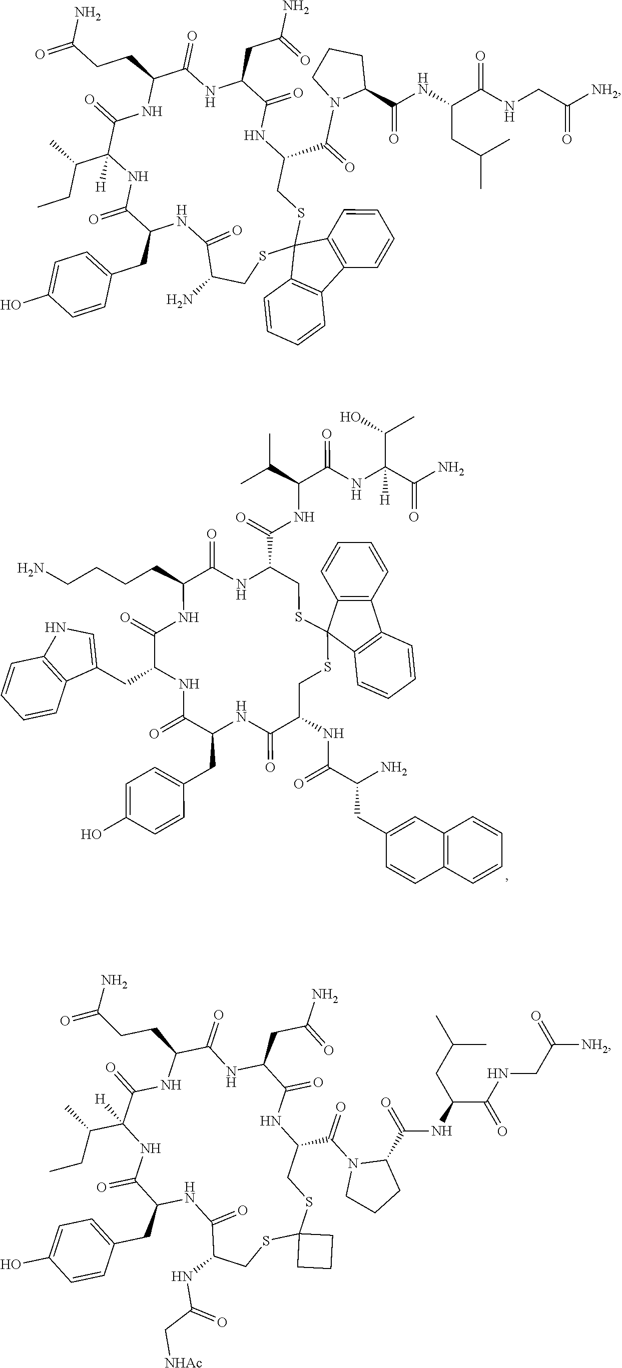

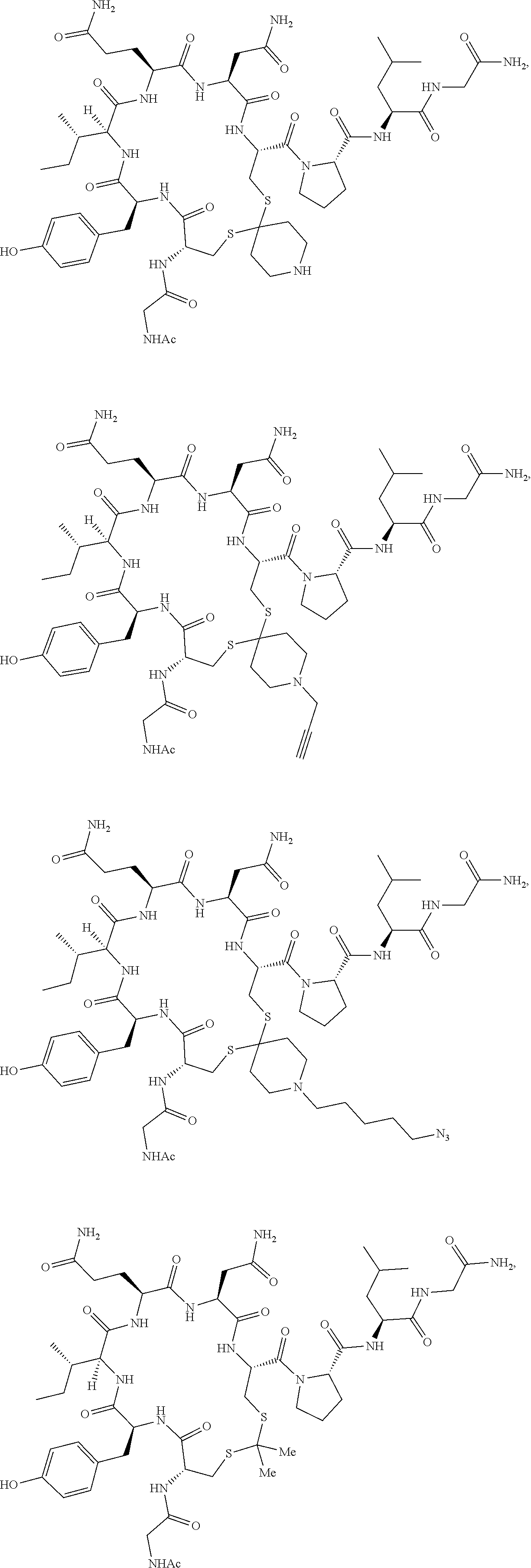

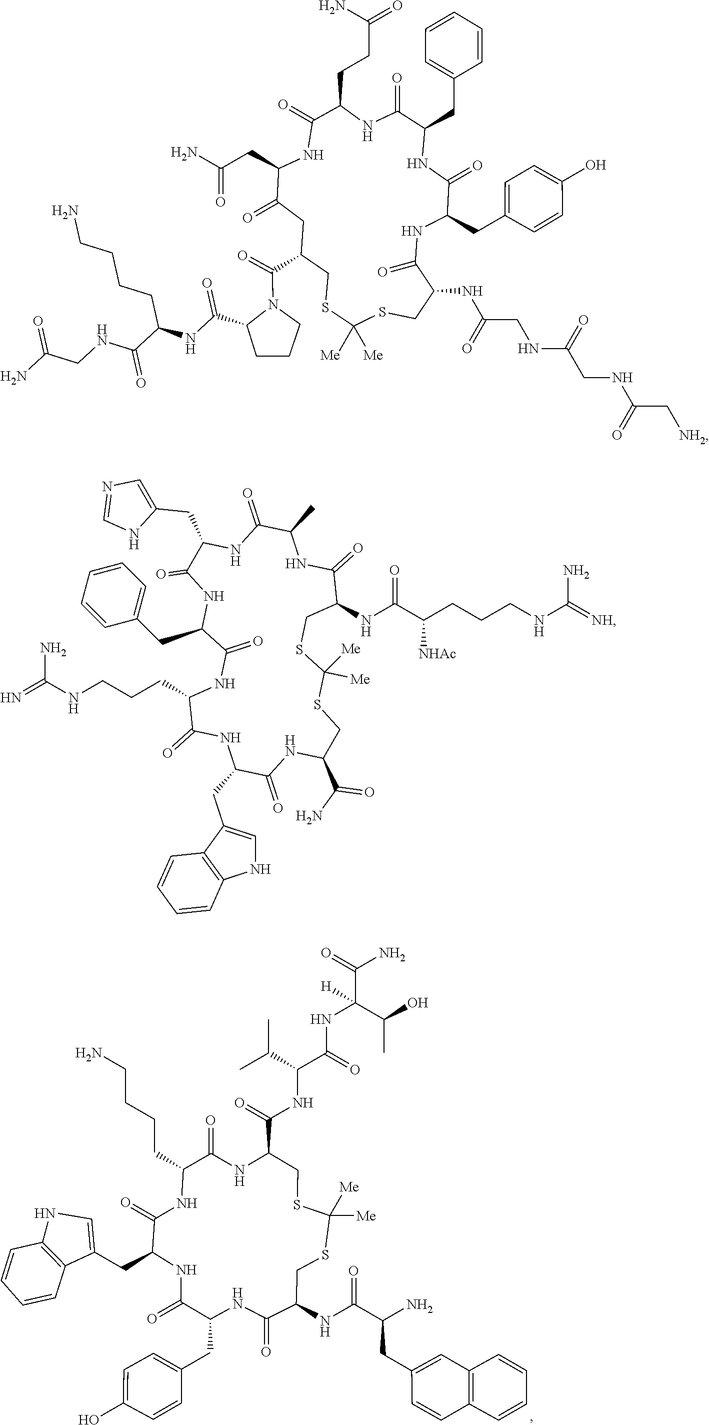

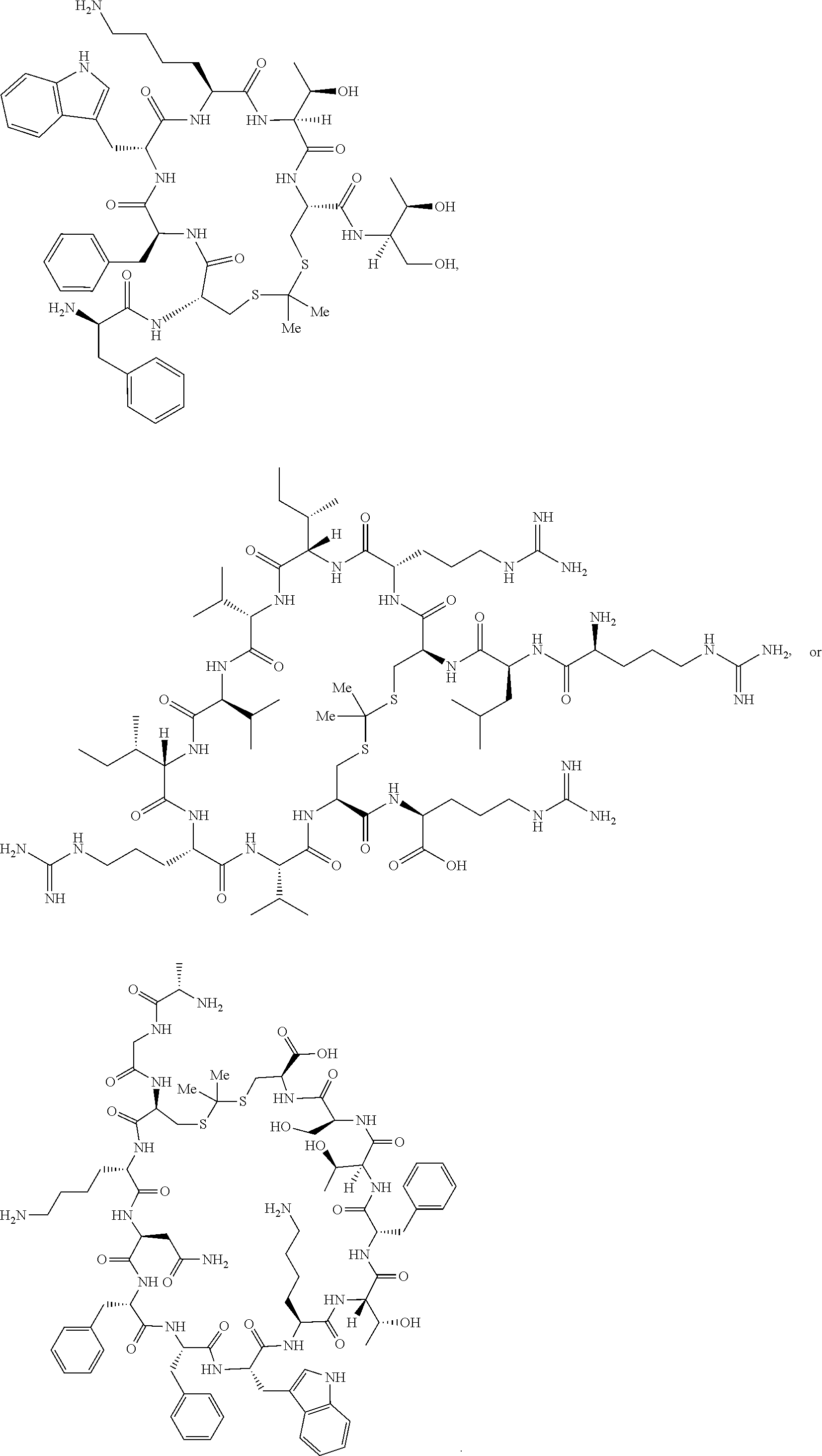

### Examples

### 1. AcHN-GCYIQNCPLG-CONH₂+9-Fluorenone (2a)

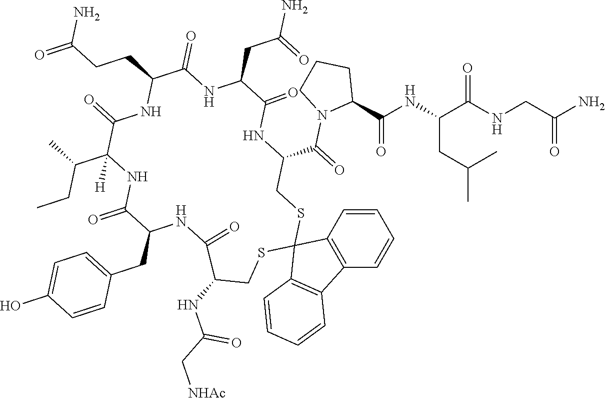 [SMILES: CC[C@H](C)[C@@H]1NC(=O)[C@H](Cc2ccc(O)cc2)NC(=O)[C@@H](NC(=O)CNC(C)=O)CSC2(SC[C@@H](C(=O)N3CCC[C@H]3C(=O)N[C@@H](CC(C)C)C(=O)NCC(N)=O)NC(=O)[C@H](CC(N)=O)NC(=O)[C@H](CCC(N)=O)NC1=O)c1ccccc1-c1ccccc12]

AcHN-GCYIQNCPLG-CONH₂+9-Fluorenone (2a)

Purified AcHN-GCYIQNCPLG-CONH₂ (1a) after SPPS (11.1 mg, 1.0 equiv) was dissolved in TFA with a final concentration of 10 mM. 9-Fluoreone (36.0 mg, 20.0 equiv) with a concentration of 200 mM was added to the solution and the reaction was stirred at room temperature around 8 h. After the reaction, the solvent was blown off under a stream of N₂, and then crude peptide product was precipitated by diethyl ether. The residue was dissolved in 10 mL 15% ACN/H₂O and subjected to preparative HPLC purification (15-60%, over 45 min). Lyophilization afforded 2a as a white powder (6.0 mg, 47%).

### 2. AcHN-CAAAC-CONH₂+9-Fluorenone (2b)

Purified AcHN-CAAAC-CONH₂ (1b) after SPPS (4.8 mg, 1.0 equiv) was dissolved in TFA with a final concentration of 10 mM. 9-Fluoreone (36.0 mg, 20.0 equiv) with a concentration of 200 mM was added to the solution and the reaction was stirred at room temperature around 8 h. After the reaction, the solvent was blown off under a stream of N₂, and then crude peptide product was precipitated by diethyl ether. The residue was dissolved in 10 mL 15% ACN/H₂O and subjected to preparative HPLC purification (15-60%, over 45 min). Lyophilization afforded 2b as a white powder (2.4 mg, 38%).

### 3. H₂N-CYFQNCPRG-CONH₂+9-Fluorenone (2c) (from Vasopressin)

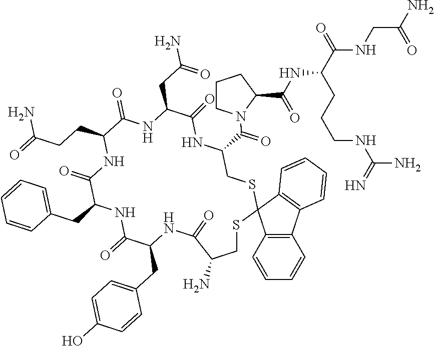 [SMILES: N=C(N)NCCC[C@H](NC(=O)[C@@H]1CCCN1C(=O)[C@@H]1CSC2(SC[C@H](N)C(=O)N[C@@H](Cc3ccc(O)cc3)C(=O)N[C@@H](Cc3ccccc3)C(=O)N[C@@H](CCC(N)=O)C(=O)N[C@@H](CC(N)=O)C(=O)N1)c1ccccc1-c1ccccc12)C(=O)NCC(N)=O]

H₂N-CYFQNCPRG-CONH₂+9-Fluorenone (2c)

Purified H₂N-CYFQNCPRG-CONH₂ (1c) after SPPS (10.8 mg, 1.0 equiv) was dissolved in TFA with a final concentration of 10 mM. 9-Fluoreone (90.0 mg, 50.0 equiv) with a concentration of 200 mM was added to the solution and the reaction was stirred at room temperature around 8 h. After the reaction, the solvent was blown off under a stream of N₂, and then crude peptide product was precipitated by diethyl ether. The residue was dissolved in 10 mL 10% ACN/H₂O and subjected to preparative HPLC purification (10-50%, over 45 min). Lyophilization afforded 2c as a white powder (3.4 mg, 27%).

### 4. H₂N-GGGCYFQNCPKG-CONH₂+9-Fluorenone (2d) (from Terlipressin)

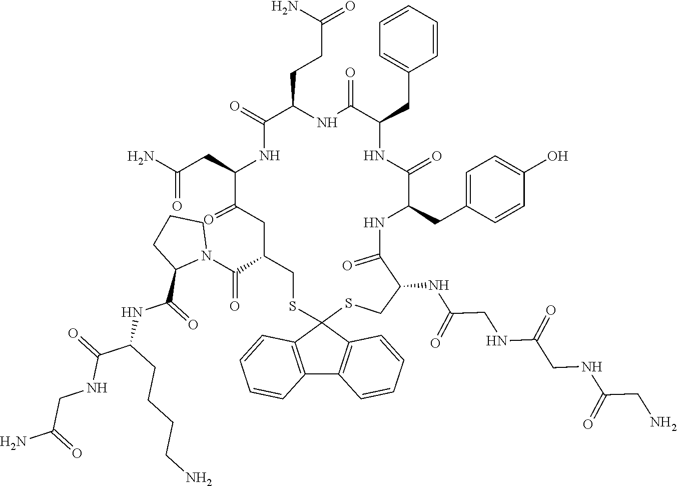 [SMILES: NCCCC[C@@H](NC(=O)[C@H]1CCCN1C(=O)[C@H]1CSC2(SC[C@@H](NC(=O)CNC(=O)CNC(=O)CN)C(=O)N[C@H](Cc3ccc(O)cc3)C(=O)N[C@H](Cc3ccccc3)C(=O)N[C@H](CCC(N)=O)C(=O)N[C@H](CC(N)=O)C(=O)C1)c1ccccc1-c1ccccc12)C(=O)NCC(N)=O]

H₂N-GGGCYFQNCPKG-CONH₂+9-Fluorenone (2d)

Purified H₂N-GGGCYFQNCPKG-CONH₂ (1d) after SPPS (12.3 mg, 1.0 equiv) was dissolved in TFA with a final concentration of 10 mM. 9-Fluoreone (36.0 mg, 20.0 equiv) with a concentration of 200 mM was added to the solution and the reaction was stirred at room temperature around 8 h. After the reaction, the solvent was blown off under a stream of N₂, and then crude peptide product was precipitated by diethyl ether. The residue was dissolved in 10 mL 10% ACN/H₂O and subjected to preparative HPLC purification (10-50%, over 45 min). Lyophilization afforded 2d as a white powder (4.0 mg, 310%).

### 5. AcHN-RC(D-)AH(D-)FRWC-CONH₂+9-Fluorenone (2e) (from Setmelanotide)

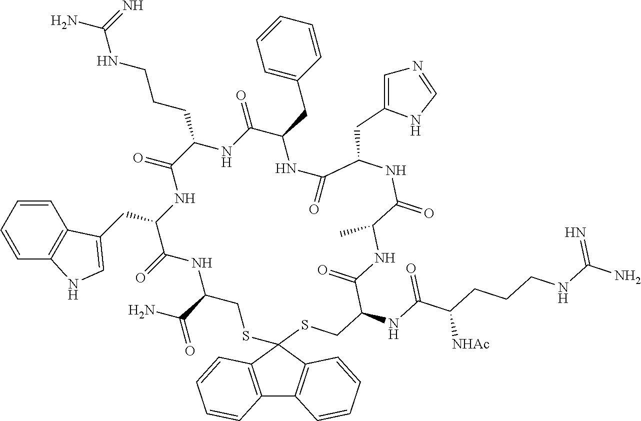

AcHN-RC(D-)AH(D-)FRWC-CONH₂+9-Fluorenone (2e)

Purified AcHN-RC(D-)AH(D-)FRWC-CONH₂ (1e) after SPPS (11.2 mg, 1.0 equiv) was dissolved in TFA with a final concentration of 10 mM. 9-Fluoreone (36.0 mg, 20.0 equiv) with a concentration of 200 mM was added to the solution and the reaction was stirred at room temperature around 8 h. After the reaction, the solvent was blown off under a stream of N₂, and then crude peptide product was precipitated by diethyl ether. The residue was dissolved in 10 mL 10% ACN/H₂O and subjected to preparative HPLC purification (10-50%, over 45 min). Lyophilization afforded 2e as a white powder (3.8 mg, 30%).

### 6. H₂N-CYIQNCPLG-CONH₂+9-Fluorenone (2f) (from Oxytocin)

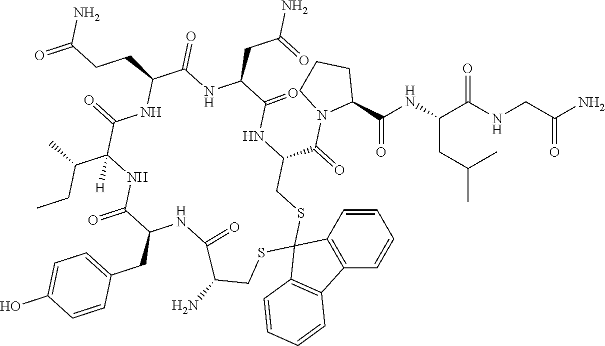 [SMILES: CC[C@H](C)[C@@H]1NC(=O)[C@H](Cc2ccc(O)cc2)NC(=O)[C@@H](N)CSC2(SC[C@@H](C(=O)N3CCC[C@H]3C(=O)N[C@@H](CC(C)C)C(=O)NCC(N)=O)NC(=O)[C@H](CC(N)=O)NC(=O)[C@H](CCC(N)=O)NC1=O)c1ccccc1-c1ccccc12]

H₂N-CYIQNCPLG-CONH₂+9-Fluorenone (2f)

Purified H₂N-CYIQNCPLG-CONH₂ (1f) after SPPS (10.1 mg, 1.0 equiv) was dissolved in TFA with a final concentration of 10 mM. 9-Fluoreone (90.0 mg, 50.0 equiv) with a concentration of 200 mM was added to the solution and the reaction was stirred at room temperature around 8 h. After the reaction, the solvent was blown off under a stream of N₂, and then crude peptide product was precipitated by diethyl ether. The residue was dissolved in 10 mL 15% ACN/H₂O and subjected to preparative HPLC purification (15-60%, over 45 min). Lyophilization afforded 2f as a white powder (4.5 mg, 38%).

### 7. H₂N-(D-)NaICY(D-)WKVCT-CONH₂+9-Fluorenone (2g) (from Lanreotide)

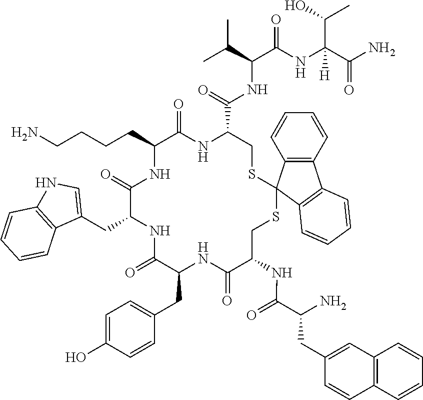 [SMILES: CC(C)[C@H](NC(=O)[C@@H]1CSC2(SC[C@H](NC(=O)[C@H](N)Cc3ccc4ccccc4c3)C(=O)N[C@@H](Cc3ccc(O)cc3)C(=O)N[C@H](Cc3c[nH]c4ccccc34)C(=O)N[C@@H](CCCCN)C(=O)N1)c1ccccc1-c1ccccc12)C(=O)N[C@H](C(N)=O)[C@@H](C)O]

H₂N-(D-)NaICY(D-)WKVCT-CONH₂+9-Fluorenone (2g)

Purified H₂N-(D-)NaICY(D-)WKVCT-CONH₂ (1g) after SPPS (10.9 mg, 1.0 equiv) was dissolved in TFA with a final concentration of 10 mM. 9-Fluoreone (90.0 mg, 50.0 equiv) with a concentration of 200 mM was added to the solution and the reaction was stirred at room temperature around 8 h. After the reaction, the solvent was blown off under a stream of N₂, and then crude peptide product was precipitated by diethyl ether. The residue was dissolved in 10 mL 20% ACN/H₂O and subjected to preparative HPLC purification (20-70%, over 45 min). Lyophilization afforded 2g as a white powder (6.6 mg, 54%).

### 8. AcHN-GCYIQNCPLG-CONH₂+cyclobutanone (2h)

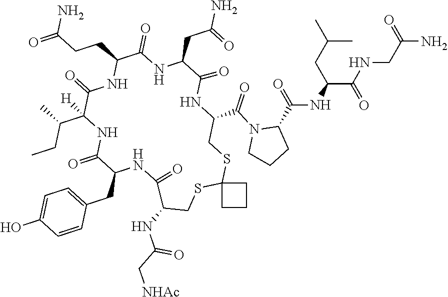 [SMILES: CC[C@H](C)[C@@H]1NC(=O)[C@H](Cc2ccc(O)cc2)NC(=O)[C@@H](NC(=O)CNC(C)=O)CSC2(CCC2)SC[C@@H](C(=O)N2CCC[C@H]2C(=O)N[C@@H](CC(C)C)C(=O)NCC(N)=O)NC(=O)[C@H](CC(N)=O)NC(=O)[C@H](CCC(N)=O)NC1=O]

AcHN-GCYIQNCPLG-CONH₂+cyclobutanone (2h)

Purified AcHN-GCYIQNCPLG-CONH₂ (1a) after SPPS (11.1 mg, 1.0 equiv) was dissolved in TFA with a final concentration of 10 mM. Cyclobutanone (14.0 mg, 20.0 equiv) with a concentration of 200 mM was added to the solution and the reaction was stirred at room temperature around 8 h. After the reaction, the solvent was blown off under a stream of N₂, and then crude peptide product was precipitated by diethyl ether. The residue was dissolved in 10 mL 15% ACN/H₂O and subjected to preparative HPLC purification (15-60%, over 45 min). Lyophilization afforded 2 h as a white powder 5.2 mg, 45%).

### 9. AcHN-GCYIQNCPLG-CONH₂+Boc-4-piperidone (2i)

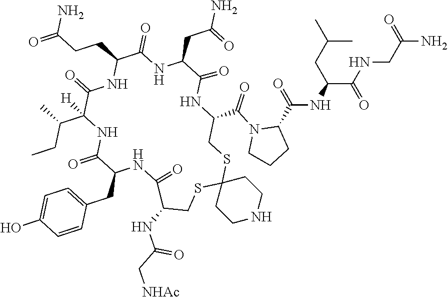 [SMILES: CC[C@H](C)[C@@H]1NC(=O)[C@H](Cc2ccc(O)cc2)NC(=O)[C@@H](NC(=O)CNC(C)=O)CSC2(CCNCC2)SC[C@@H](C(=O)N2CCC[C@H]2C(=O)N[C@@H](CC(C)C)C(=O)NCC(N)=O)NC(=O)[C@H](CC(N)=O)NC(=O)[C@H](CCC(N)=O)NC1=O]

AcHN-GCYIQNCPLG-CONH₂+Boc-4-piperidone (2i)

Purified AcHN-GCYIQNCPLG-CONH₂ (1a) after SPPS (11.1 mg, 1.0 equiv) was dissolved in TFA with a final concentration of 10 mM. N-(tert-butoxycarbonyl)-4-piperidone (39.8 mg, 20.0 equiv) with a concentration of 200 mM was added to the solution and the reaction was stirred at room temperature around 8 h. After the reaction, the solvent was blown off under a stream of N₂, and then crude peptide product was precipitated by diethyl ether. The residue was dissolved in 10 mL 10% ACN/H₂O and subjected to preparative HPLC purification (10-50%, over 45 min). Lyophilization afforded 2i as a white powder (2.3 mg, 20%).

### 10. AcHN-GCYIQNCPLG-CONH₂+1-(prop-2-yn-1-yl) piperidin-4-one (2j)

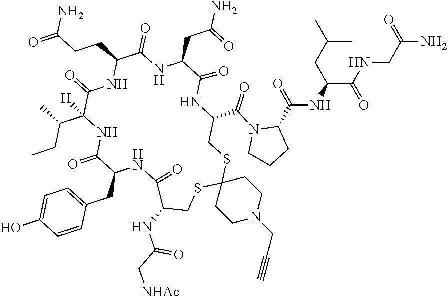 [SMILES: C#CCN1CCC2(CC1)SC[C@H](NC(=O)CNC(C)=O)C(=O)N[C@@H](Cc1ccc(O)cc1)C(=O)N[C@@H]([C@@H](C)CC)C(=O)N[C@@H](CCC(N)=O)C(=O)N[C@@H](CC(N)=O)C(=O)N[C@H](C(=O)N1CCC[C@H]1C(=O)N[C@@H](CC(C)C)C(=O)NCC(N)=O)CS2]

AcHN-GCYIQNCPLG-CONH₂+1-(prop-2-yn-1-yl) piperidin-4-one (2j)

Purified AcHN-CAAAC-CONH₂ (1a) after SPPS (11.1 mg, 1.0 equiv) was dissolved in TFA with a final concentration of 10 mM. 1-(Prop-2-yn-1-yl) piperidin-4-one (42.0 mg, 20.0 equiv) with a concentration of 200 mM was added to the solution and the reaction was stirred at room temperature around 8 h. After the reaction, the solvent was blown off under a stream of N₂, and then crude peptide product was precipitated by diethyl ether. The residue was dissolved in 10 mL 15% ACN/H₂O and subjected to preparative HPLC purification (15-60%, over 45 min). Lyophilization afforded 2j as a white powder (5.6 mg, 46%).

### 11. AcHN-GCYIQNCPLG-CONH₂+5-Azidopentyl) piperidin-4-one (2k)

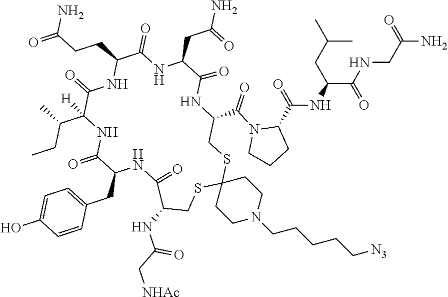 [SMILES: CC[C@H](C)[C@@H]1NC(=O)[C@H](Cc2ccc(O)cc2)NC(=O)[C@@H](NC(=O)CNC(C)=O)CSC2(CCN(CCCCCN=[N+]=[N-])CC2)SC[C@@H](C(=O)N2CCC[C@H]2C(=O)N[C@@H](CC(C)C)C(=O)NCC(N)=O)NC(=O)[C@H](CC(N)=O)NC(=O)[C@H](CCC(N)=O)NC1=O]

AcHN-GCYIQNCPLG-CONH₂+5-Azidopentyl) piperidin-4-one (2k)

Purified 11 AcHN-GCYIQNCPLG-CONH₂ (1a) after SPPS (11.1 mg, 1.0 equiv) was dissolved in TFA with a final concentration of 10 mM. 1-(5-Azidopentyl) piperidin-4-one (42.0 mg, 20.0 equiv) with a concentration of 200 mM was added to the solution and the reaction was stirred at room temperature around 8 h. After the reaction, the solvent was blown off under a stream of N₂, and then crude peptide product was precipitated by diethyl ether. The residue was dissolved in 10 mL 10% ACN/H₂O and subjected to preparative HPLC purification (10-50%, over 45 min). Lyophilization afforded 2k as a white powder (5.6 mg, 43%).

### 12. AcHN-GCYIQNCPLG-CONH₂+Acetone (3a)

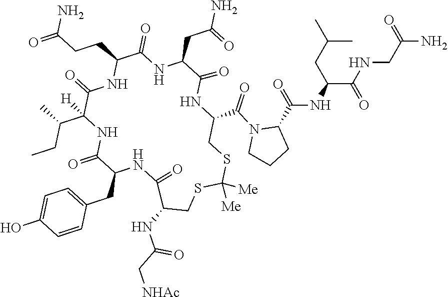 [SMILES: CC[C@H](C)[C@@H]1NC(=O)[C@H](Cc2ccc(O)cc2)NC(=O)[C@@H](NC(=O)CNC(C)=O)CSC(C)(C)SC[C@@H](C(=O)N2CCC[C@H]2C(=O)N[C@@H](CC(C)C)C(=O)NCC(N)=O)NC(=O)[C@H](CC(N)=O)NC(=O)[C@H](CCC(N)=O)NC1=O]

AcHN-GCYIQNCPLG-CONH₂+Acetone (3a)

Purified AcHN-GCYIQNCPLG-CONH₂ (1a) after SPPS (11.1 mg, 1.0 equiv) was dissolved in a mixture solvent (1.0 mL) of TFA and acetone (v:v=1:1) with a final concentration of 10 mM. The reaction was stirred at room temperature around 8 h. After the reaction, the solvent was blown off under a stream of N₂, and then crude peptide product was precipitated by diethyl ether. The residue was dissolved in 10 mL 15% ACN/H₂O and subjected to preparative HPLC purification (10-50%, over 35 min). Lyophilization afforded 3a as a white powder (6.6 mg, 63%).

### 13. H₂N-GGGCYFQNCPKG-CONH₂+Acetone (3b) (from Terlipressin)

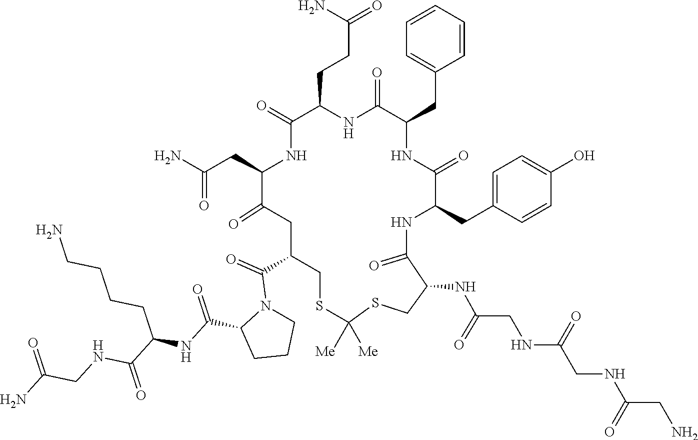 [SMILES: CC1(C)SC[C@H](C(=O)N2CCC[C@@H]2C(=O)N[C@H](CCCCN)C(=O)NCC(N)=O)CC(=O)[C@@H](CC(N)=O)NC(=O)[C@@H](CCC(N)=O)NC(=O)[C@@H](Cc2ccccc2)NC(=O)[C@@H](Cc2ccc(O)cc2)NC(=O)[C@H](NC(=O)CNC(=O)CNC(=O)CN)CS1]

H₂N-GGGCYFQNCPKG-CONH₂+Acetone (3b)

Purified H₂N-GGGCYFQNCPKG-CONH₂ (1d) after SPPS (12.3 mg, 1.0 equiv) was dissolved in a mixture solvent (1.0 mL) of TFA and acetone (v:v=1:1) with a final concentration of 10 mM. The reaction was stirred at room temperature around 8 h. After the reaction, the solvent was blown off under a stream of N₂, and then crude peptide product was precipitated by diethyl ether. The residue was dissolved in 10 mL 10% ACN/H₂O and subjected to preparative HPLC purification (10-50%, over 35 min). Lyophilization afforded 3b as a white powder (8.3 mg, 65%).

### 14. AcHN-RC(D-)AH(D-)FRWC-CONH₂+Acetone (3c) (from Setmelanotide)

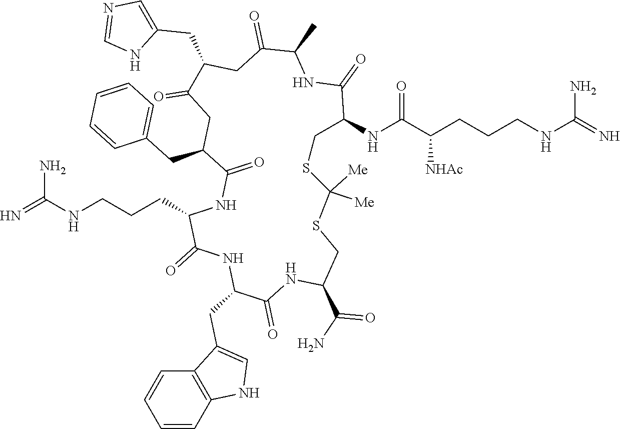 [SMILES: CC(=O)N[C@@H](CCCNC(=N)N)C(=O)N[C@H]1CSC(C)(C)SC[C@@H](C(N)=O)NC(=O)[C@H](Cc2c[nH]c3ccccc23)NC(=O)[C@H](CCCNC(=N)N)NC(=O)[C@@H](Cc2ccccc2)CC(=O)[C@H](Cc2cnc[nH]2)CC(=O)[C@@H](C)NC1=O]

AcHN-RC(D-)AH(D-)FRWC-CONH₂+Acetone (3c)

Purified AcHN-RC(D-)AH(D-)FRWC-CONH₂ (1e) after SPPS (11.9 mg, 1.0 equiv) was dissolved in a mixture solvent (1.0 mL) of TFA and acetone (v:v=1:1) with a final concentration of 10 mM. The reaction was stirred at room temperature around 8 h. After the reaction, the solvent was blown off under a stream of N₂, and then crude peptide product was precipitated by diethyl ether. The residue was dissolved in 10 mL 15% ACN/H₂O and subjected to preparative HPLC purification (10-60%, over 35 min). Lyophilization afforded 3c as a white powder (8.9 mg, 77%).

### 15. H₂N-(D-)NaICY(D-)WKVCT-CONH₂+Acetone (3d) (from Lanreotide)

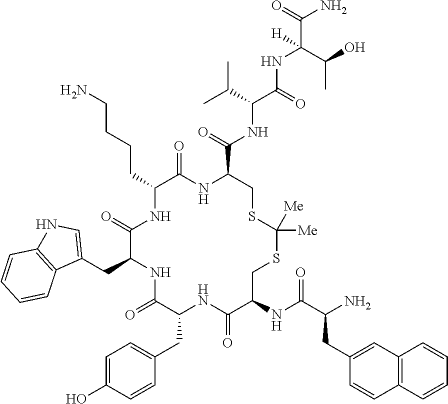 [SMILES: CC(C)[C@@H](NC(=O)[C@H]1CSC(C)(C)SC[C@@H](NC(=O)[C@@H](N)Cc2ccc3ccccc3c2)C(=O)N[C@H](Cc2ccc(O)cc2)C(=O)N[C@@H](Cc2c[nH]c3ccccc23)C(=O)N[C@H](CCCCN)C(=O)N1)C(=O)N[C@@H](C(N)=O)[C@H](C)O]

H₂N-(D-)NaICY(D-)WKVCT-CONH₂+Acetone (3d)

Purified H₂N-(D-)NaI CY(D-)WKVCT-CONH₂ (1g) after SPPS (10.9 mg, 1.0 equiv) was dissolved in a mixture solvent (1.0 mL) of TFA and acetone (v:v=1:1) with a final concentration of 10 mM. The reaction was stirred at room temperature around 8 h. After the reaction, the solvent was blown off under a stream of N₂, and then crude peptide product was precipitated by diethyl ether. The residue was dissolved in 10 mL 20% ACN/H₂O and subjected to preparative HPLC purification (20-70%, over 35 min). Lyophilization afforded 3e as a white powder (5.3 mg, 47%).

### 16. H₂N-(D-)FCF(D-)WKTCT-CH₂OH+Acetone (3e) (from Octreotide)

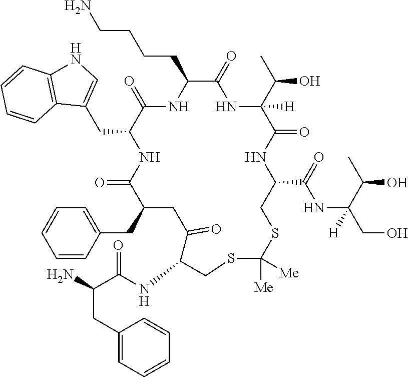 [SMILES: C[C@@H](O)[C@@H]1NC(=O)[C@H](CCCCN)NC(=O)[C@@H](Cc2c[nH]c3ccccc23)NC(=O)[C@H](Cc2ccccc2)CC(=O)[C@@H](NC(=O)[C@H](N)Cc2ccccc2)CSC(C)(C)SC[C@@H](C(=O)N[C@H](CO)[C@@H](C)O)NC1=O]

H₂N-(D-)FCF(D-)WKTCT-CH₂OH+Acetone (3e)

Purified H₂N-(D-)FCF(D-)WKTCT-CH₂OH (1h) after SPPS (10.6 mg, 1.0 equiv) was dissolved in a mixture solvent (1.0 mL) of TFA and acetone (v:v=1:1) with a final concentration of 10 mM. The reaction was stirred at room temperature around 8 h. After the reaction, the solvent was blown off under a stream of N₂, and then crude peptide product was precipitated by diethyl ether. The residue was dissolved in 10 mL 15% ACN/H₂O and subjected to preparative HPLC purification (15-60%, over 35 min). Lyophilization afforded 3e as a white powder (7.5 mg, 71%).

### 17. H₂N-RLCRIVVIRVCR-COOH+Acetone (3f) (from Bactenecin)

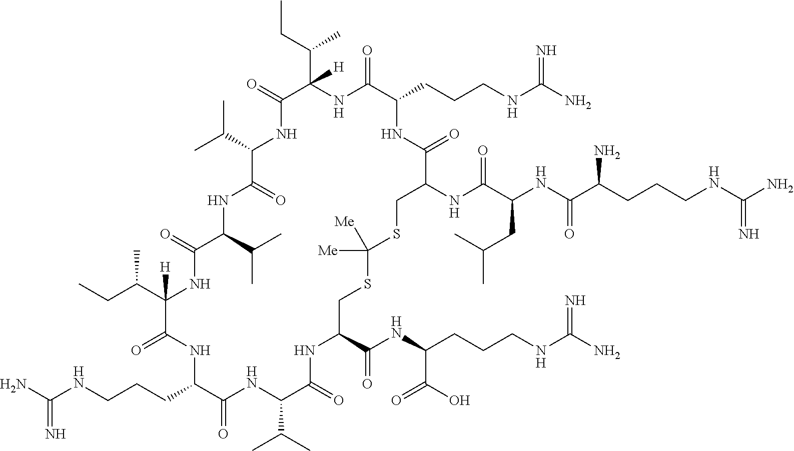 [SMILES: CC[C@H](C)[C@@H]1NC(=O)[C@H](CCCNC(=N)N)NC(=O)C(NC(=O)[C@H](CC(C)C)NC(=O)[C@@H](N)CCCNC(=N)N)CSC(C)(C)SC[C@@H](C(=O)N[C@@H](CCCNC(=N)N)C(=O)O)NC(=O)[C@H](C(C)C)NC(=O)[C@H](CCCNC(=N)N)NC(=O)[C@H]([C@@H](C)CC)NC(=O)[C@H](C(C)C)NC(=O)[C@H](C(C)C)NC1=O]

H₂N-RLCRIVVIRVCR-COOH+Acetone (3f)

Purified H₂N-RLCRIVVIRVCR-COOH (1i) after SPPS (7.4 mg, 1.0 equiv) was dissolved in a mixture solvent (10.0 mL) of TFA and acetone (v:v=1:1) with a final concentration of 0.5 mM. The reaction was stirred at room temperature around 8 h. After the reaction, the solvent was blown off under a stream of N₂, and then crude peptide product was precipitated by diethyl ether. The residue was dissolved in 10 mL 10% ACN/H₂O and subjected to preparative HPLC purification (10-60%, over 35 min). Lyophilization afforded 3f as a white powder (1.1 mg, 15%).

### 18. H₂N-AGCKNFFWKTFTSC-CONH₂+Acetone (3g) (from Somatostatin)

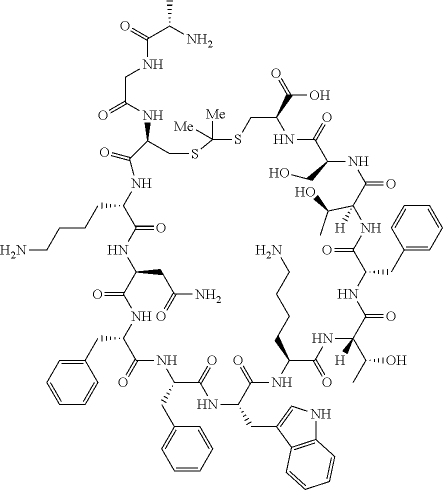 [SMILES: C[C@H](N)C(=O)NCC(=O)N[C@H]1CSC(C)(C)SC[C@@H](C(=O)O)NC(=O)[C@H](CO)NC(=O)[C@H]([C@@H](C)O)NC(=O)[C@H](Cc2ccccc2)NC(=O)[C@H]([C@@H](C)O)NC(=O)[C@H](CCCCN)NC(=O)[C@H](Cc2c[nH]c3ccccc23)NC(=O)[C@H](Cc2ccccc2)NC(=O)[C@H](Cc2ccccc2)NC(=O)[C@H](CC(N)=O)NC(=O)[C@H](CCCCN)NC1=O]

H₂N-AGCKNFFWKTFTSC-CONH₂+Acetone (3g)

Purified H₂N-AGCKNFFWKTFTSC-CONH₂ (4j) after SPPS (16.4 mg, 1.0 equiv) was dissolved in a mixture solvent (1.0 mL) of TFA and acetone (v:v=1:1) with a final concentration of 10 mM. The reaction was stirred at room temperature around 8 h. After the reaction, the solvent was blown off under a stream of N₂, and then crude peptide product was precipitated by diethyl ether. The residue was dissolved in 10 mL 20% ACN/H₂O and subjected to preparative HPLC purification (20-70%, over 35 min). Lyophilization afforded 3g as a white powder (8.6 mg, 51%).

It is understood that the disclosed method and compositions are not limited to the particular methodology, protocols, and reagents described as these can vary. It is also to be understood that the terminology used herein is for the purpose of describing particular embodiments only and is not intended to limit the scope of the present invention which will be limited only by the appended claims.

Those skilled in the art will recognize or be able to ascertain using no more than routine experimentation, many equivalents to the specific embodiments of the method and compositions described herein. Such equivalents are intended to be encompassed by the following claims.

1. A method of late-stage cyclization of a peptide comprising:
maintaining a reaction mixture at a sufficient temperature for a sufficient period of time to form a product,
wherein the reaction mixture comprises the peptide comprising two or more cysteine residues, a cyclic ketone reagent or acetone, and a solvent,
wherein the product comprises a thioacetalated peptide, and the thioacetalation couples two of the cysteine residues of the peptide.

2. The method of claim 1, wherein the peptide is linear or cyclic (including monocyclic, bicyclic, etc.)

3. The method of claim 1, wherein the peptide is a random peptide or a peptide drug.

4. The method of claim 1, wherein the peptide comprises natural amino acids.

5. The method of claim 1, wherein the cyclic ketone reagent is a cyclic ketone reagent having the structure of: 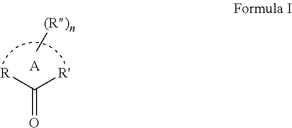
wherein R and R′ are independently an alkyl and [symbol] is absent, or R and R′ together form a cyclic moiety A,
wherein A is a cycloalkyl, a cycloalkenyl, a cycloalkynyl, an aryl, a polyaryl, a heteroaryl, a heteropolyaryl, or a heterocyclic,
wherein R″ represents hydrogen or a substituent on the cyclic moiety A, and each occurrence of R″ is independently a benzyl, an allyl, an alkyl (such as a C₁-C₆ alkyl), a halogen, —CN, —CF₃, —NO₂, an alkoxy, or an aryl, and
wherein n is an integer from 0 to 10, from 0 to 8, from 0 to 6, from 0 to 4, or from 0 to 2.

6. The method of claim 5, wherein R and R′ are independently an alkyl, such as methyl.

7. The method of claim 5, wherein A is cyclobutane, azetidine, cyclopentane, fluorenone, cyclohexane, or piperidine.

8. The method of claim 1, wherein the solvent is trifluoroacetic acid.

9. The method of claim 8, wherein trifluoroacetic acid acts as catalyst and is the only solvent in the reaction mixture.

10. The method of claim 1, wherein the reaction mixture is maintained at a temperature ranging from 20° C. to 35° C., such as about 30° C., for a time period up to 1 hour, up to 2 hours, up to 3 hours, ranging from 10 mins to 1 hour, from 20 mins to 2 hours, or from 30 mins to 3 hours.

11. The method of claim 1, wherein the peptide and the cyclic ketone reagent have a molar ratio (peptide:cyclic ketone reagent) ranging from 0.1 to 1, such as about 0.2.

12. The method of claim 1, wherein the cyclic ketone reagent has any one of the following structures: 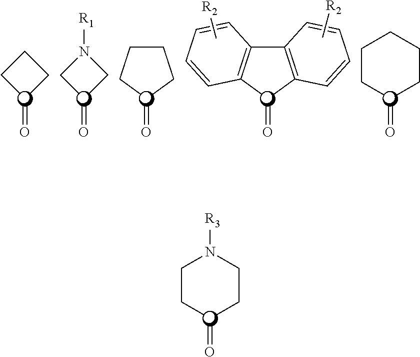
wherein R¹ and R³ are an H, a benzyl, an allyl, an alkyl (such as a C1-C6 alkyl), and
wherein each R² is independently an H, a benzyl, an allyl, an alkyl (such as a C1-C6 alkyl), a halogen, —CN, —CF₃, —NO₂, an alkoxy, or an aryl.

13. The method of claim 1, wherein the thioacetalated peptide has any one of the following structures: 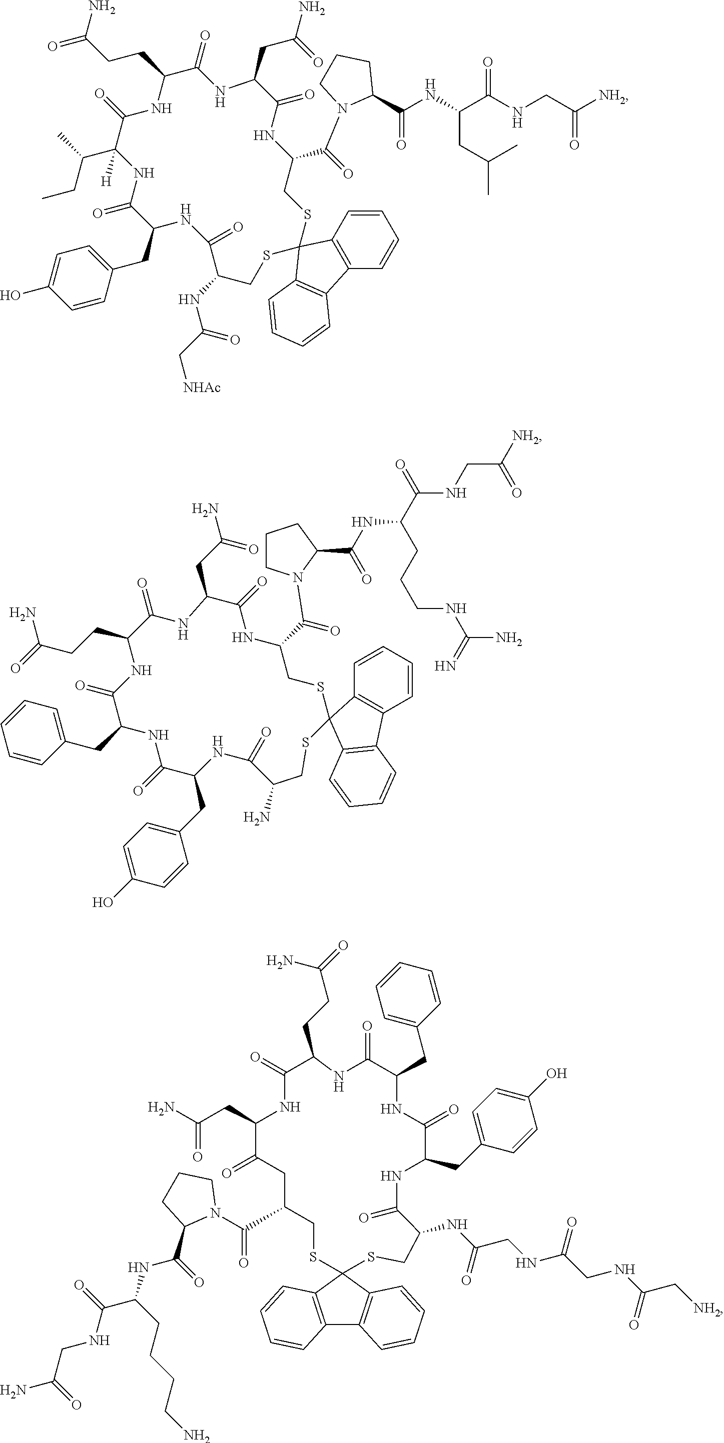 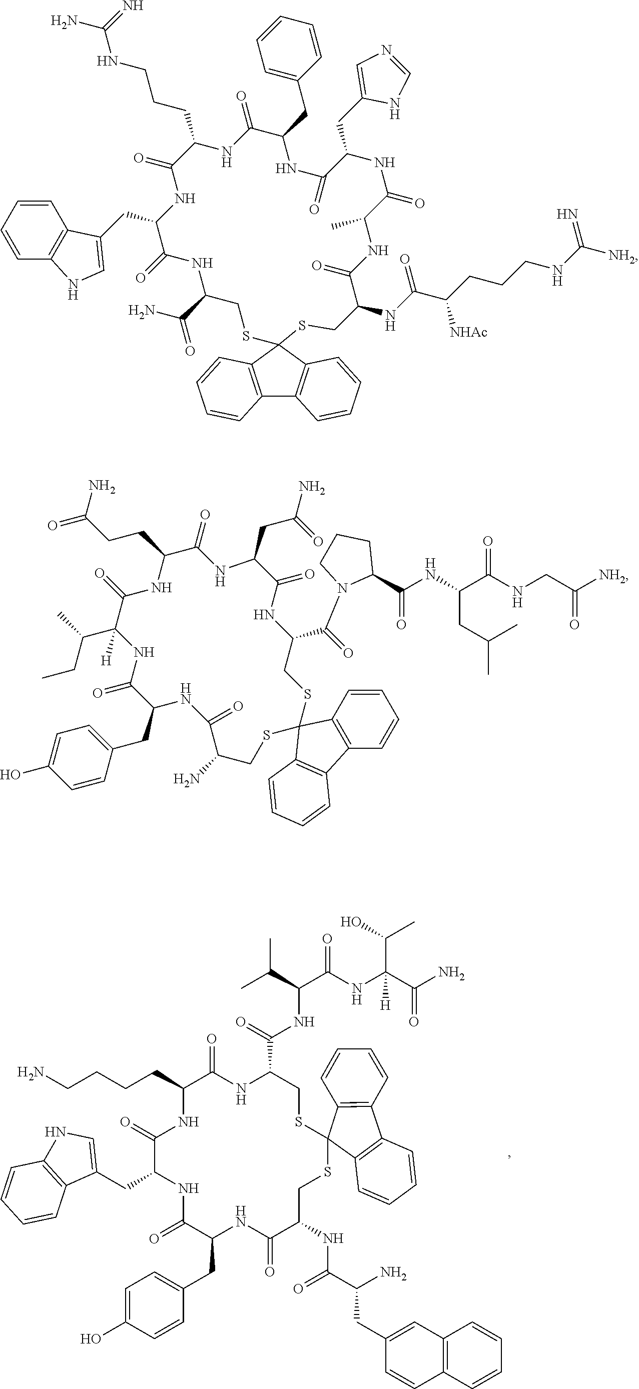 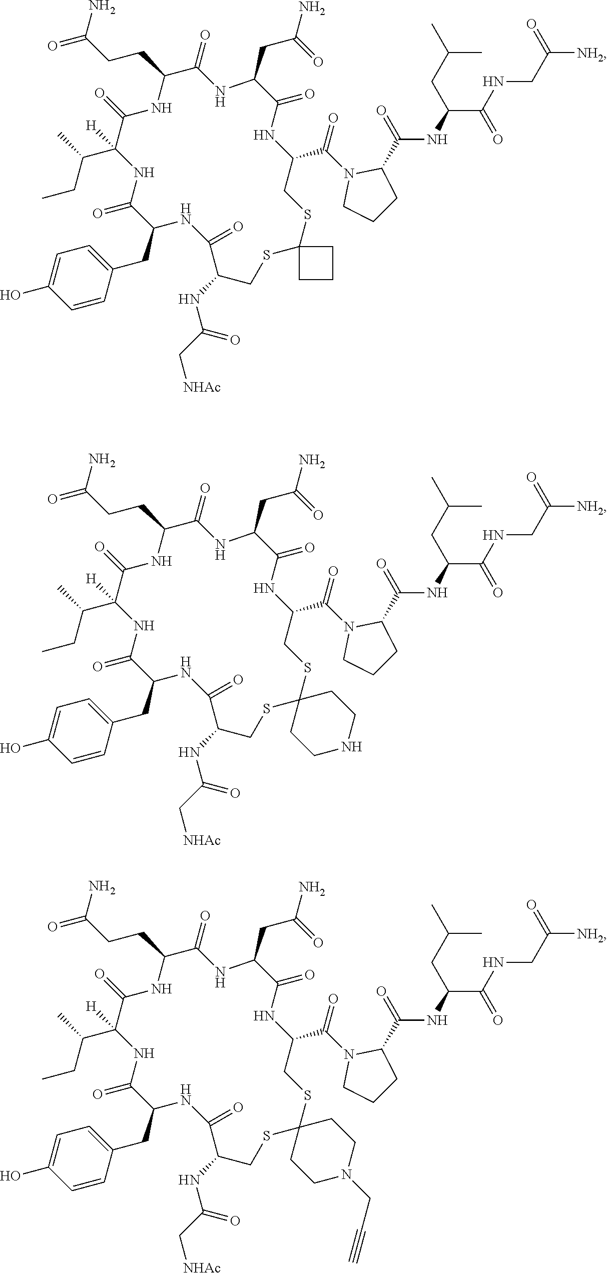 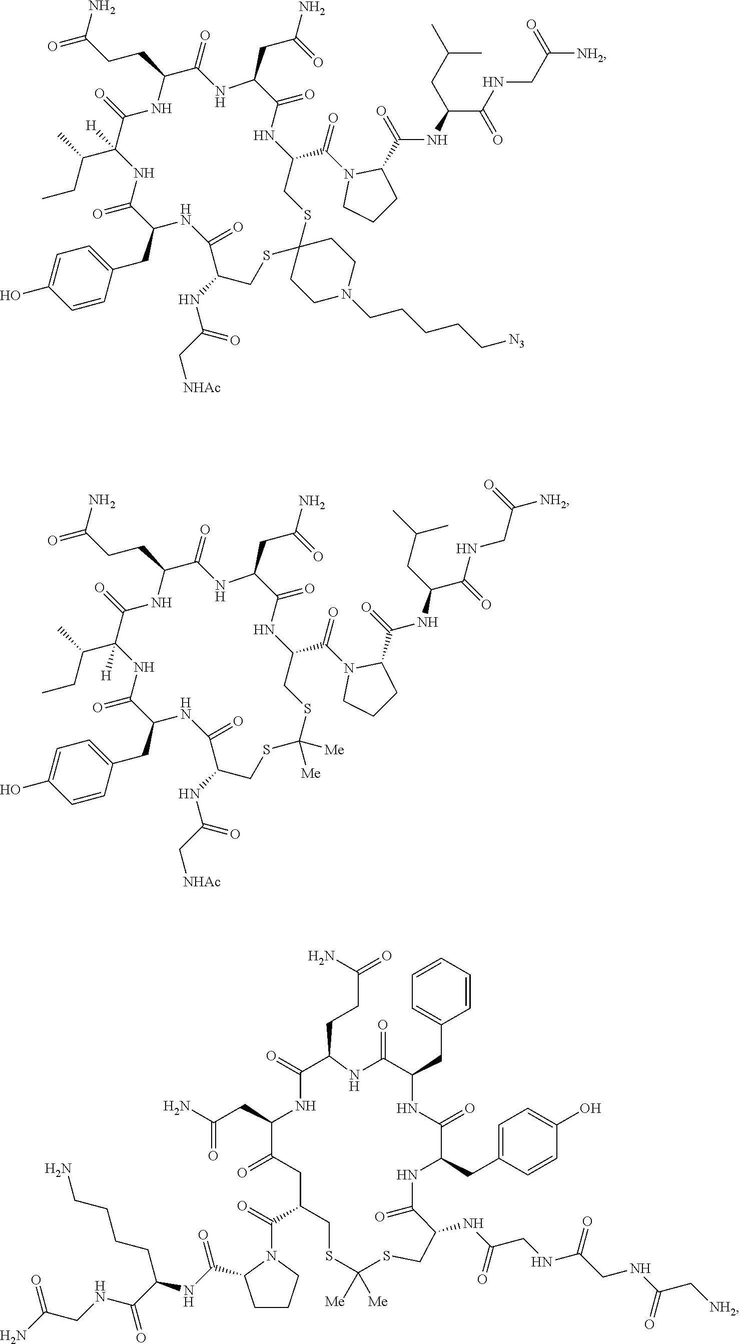 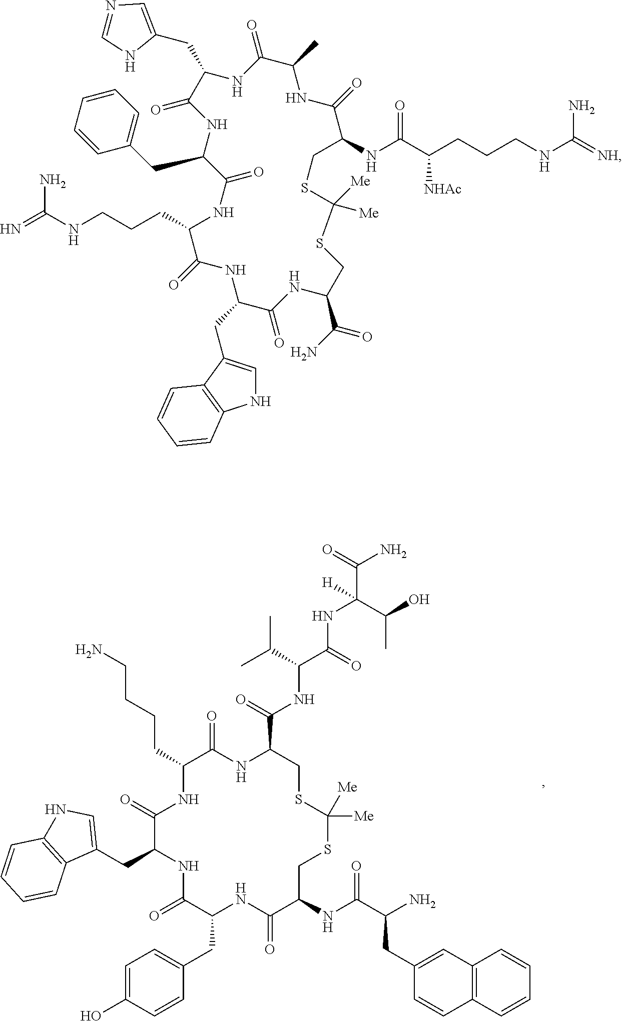 [SMILES: CC(=O)N[C@@H](CCCNC(=N)N)C(=O)N[C@H]1CSC(C)(C)SC[C@@H](C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2cnc[nH]2)NC(=O)[C@@H](C)NC1=O ; CC(C)[C@@H](NC(=O)[C@H]1CSC(C)(C)SC[C@@H](NC(=O)[C@@H](N)Cc2ccc3ccccc3c2)C(=O)N[C@H](Cc2ccc(O)cc2)C(=O)N[C@@H](Cc2c[nH]c3ccccc23)C(=O)N[C@H](CCCCN)C(=O)N1)C(=O)N[C@@H](C(N)=O)[C@H](C)O] 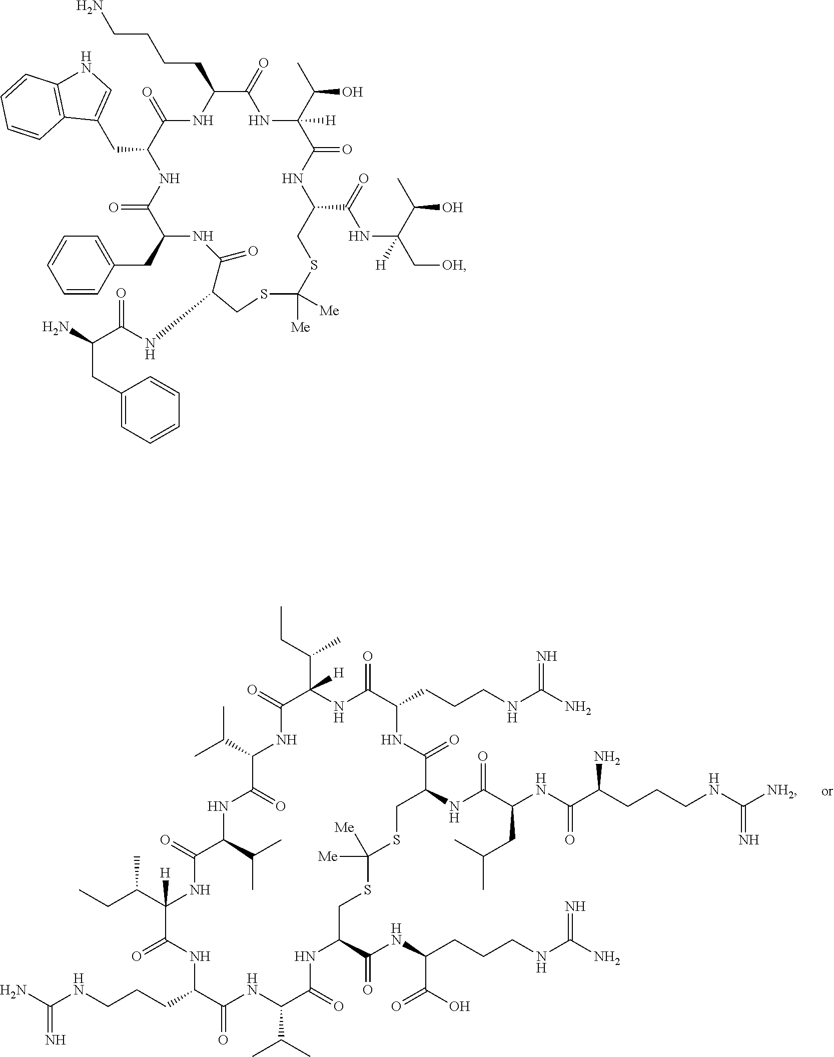 [SMILES: C[C@@H](O)[C@@H]1NC(=O)[C@H](CCCCN)NC(=O)[C@@H](Cc2c[nH]c3ccccc23)NC(=O)[C@H](Cc2ccccc2)NC(=O)[C@@H](NC(=O)[C@H](N)Cc2ccccc2)CSC(C)(C)SC[C@@H](C(=O)N[C@H](CO)[C@@H](C)O)NC1=O ; CC[C@H](C)[C@@H]1NC(=O)[C@H](CCCNC(=N)N)NC(=O)[C@@H](NC(=O)[C@H](CC(C)C)NC(=O)[C@@H](N)CCCNC(=N)N)CSC(C)(C)SC[C@@H](C(=O)N[C@@H](CCCNC(=N)N)C(=O)O)NC(=O)[C@H](C(C)C)NC(=O)[C@H](CCCNC(=N)N)NC(=O)[C@H]([C@@H](C)CC)NC(=O)[C@H](C(C)C)NC(=O)[C@H](C(C)C)NC1=O] 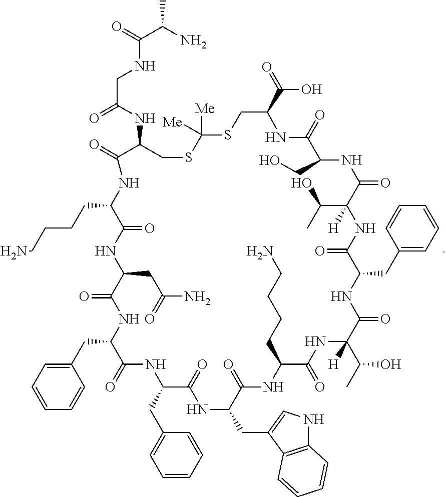 [SMILES: C[C@H](N)C(=O)NCC(=O)N[C@H]1CSC(C)(C)SC[C@@H](C(=O)O)NC(=O)[C@H](CO)NC(=O)[C@H]([C@@H](C)O)NC(=O)[C@H](Cc2ccccc2)NC(=O)[C@H]([C@@H](C)O)NC(=O)[C@H](CCCCN)NC(=O)[C@H](Cc2c[nH]c3ccccc23)NC(=O)[C@H](Cc2ccccc2)NC(=O)[C@H](Cc2ccccc2)NC(=O)[C@H](CC(N)=O)NC(=O)[C@H](CCCCN)NC1=O]
